(19) United States
(12) Patent Application Publication
(10) Pub. No.: US 2014/0343460 A1
(43) Pub. Date: Nov. 20, 2014

(54) MOBILE GAIT FORCE AND MOTION ANALYSIS SYSTEM

(71) Applicant: UT-BATTELLE, LLC, Oak Ridge, TN (US)

(72) Inventors: Boyd McCutchen EVANS, III, Oak Ridge, TN (US); John Kyle P. MUELLER, Warsaw, IN (US); Milton Nance ERICSON, Knoxville, TN (US); Ethan FARQUHAR, Knoxville, TN (US)

(21) Appl. No.: 13/894,457

(22) Filed: May 15, 2013

(51) Int. Cl. A61B 5/11; A61B 5/103; A61B 5/00

(52) U.S. Cl. CPC A61B 5/112; A61B 5/1121; A61B 5/725; A61B 5/1038; A61B 5/6829; A61B 5/4851
USPC 600/595

(57) ABSTRACT

Systems and methods for performing gait analysis of a subject are provided. A method includes obtaining a plurality of measurement sets for a subject, each of the plurality of measurement sets including inertial measurements obtained from a sensor device associated with a different one of a plurality of segments of the subject and calculating a sensor orientation for the sensor device associated with each of the plurality of segments based at least on a portion of a corresponding one the plurality of measurement sets. The method also includes computing an estimate of a segment orientation for each of the plurality of segments based on a data fusion process applied at each of the plurality of segments, where the data fusion process includes combining at least a one of the plurality measurement sets and the corresponding sensor orientation to estimate the segment orientation.

### GOVERNMENT LICENSE RIGHTS

This invention was made with government support under contract No. 2095-V215-10 awarded by the U.S. Army Medical Material Command and contract No. DE-AC05-00OR22725 awarded by the Department of Energy. The government has certain rights in the invention.

### FIELD OF THE INVENTION

The various embodiments relate to methods for quantifying biomechanical data, and more specifically to biomechanical data related to a mobile gait force associated with a lower limb prosthesis.

### BACKGROUND

American military healthcare system has improved treatment of combat casualties—increase from 76% to 87% survival since Vietnam. As a result of these improvements in survivability, there has been an influx of soldiers dealing with lifelong major injuries attributed to roadside bombs and Improvised Explosive Devises (IEDs). For example, such injuries result in the amputation (transtibial or transfemoral) of a lower limb for a large number of soldiers. In addition to such military amputees, there are also about 100,000 lower limb amputations occurring per year in the general population. As a result, many of these patients are typically fitted with lower limb prostheses. In the case of military personnel, these individuals are typically otherwise healthy and the lower limb prosthesis allows them to engage in an active lifestyle.

However, lower limb prosthetic patients are generally at risk for injury and chronic disease in their unaffected leg, including their healthy joints. A culprit for this increased for injury and chronic disease in unaffected legs and healthy joints is the asymmetric gait generally resulting from the use of some prosthetic lower limb devices. An asymmetric gait can cause increased joint loading and higher energy consumption. Such increased energy consumption can limit the amputee's participation in various activities and work functions. More importantly, if injuries and chronic diseases develop in the unaffected leg or healthy joints, this may permanently limit the amputee's ability to function. Table 1 summarizes peak ground reaction force between intact and prosthetic legs in transtibial amputees at various speeds:

TABLE 1

| Speed | Prosthetic legs | Intact legs | |
|---|---|---|---|
| (m/s) | Fz (BW) | Fz (BW) | ASI (%) |
| 0.5 | 1.16 (0.07) | 1.40 (0.23) | 17.54 (19.40) |
| 0.9 | 1.17 (0.05) | 1.40 (0.23) | 16.96 (18.49) |
| 1.2 | 1.18 (0.08) | 1.47 (0.20) | 22.59 (18.44) |
| Max | 1.23 (0.08) | 1.68 (0.32) | 29.34 (19.70) |

(From L. Nolan et al., “Adjustments in gait symmetry with walking speed in transfemoral and trans-tibial amputees.” Gait and Posture 17 (2003) 142-151.) For at least these reasons, a need exists to limit asymmetric gait by better fitting of prosthetic devices and training or monitoring of patients in the use of said devices.

Typically, fitting is performed by a trained prosthetist or physical therapist during one or more office visits. Prosthetists have training and experience that allows them to adjust the prosthetic based on feedback from the patient and their observation of the patient's gait. This type of traditional fitting is typically an iterative process requiring multiple sessions. During a session, the prosthetist makes observations to determine the required adjustments. First, the patient walks for an interval of time while the prosthetist observes. The prosthetist then makes an adjustment, and the process is repeated until the prosthetist and the patient are satisfied with the operation of the prosthesis. In most prosthetic devices, the adjustments can be made to a stump/device interface, any 6-degrees of freedom (DOF) joints or adapters, a prosthetic foot, or any combination thereof.

In general, the data collected is typically limited to the prosthetist's observations, which are, of course subjective and variable from one prosthetist to another. This subjectivity can lead to inconsistent results across the plurality of patients and also to counterproductive results if one patient visits a plurality of prosthetists.

In some cases, it is possible to obtain quantifiable biomechanical data for a patient's gait in a so-called “gait laboratory.” Unfortunately, the process of obtaining biomechanical data in a gait laboratory is expensive and time-consuming. Accordingly, such methods are not generally available to most prosthetists and therapists. Additionally, a problem exists in translating any quantifiable biomechanical data collected in a gait laboratory into clinically meaningful data which can be used by a prosthetist or a therapist to make actual adjustments to prostheses.

Therefore, a need exists for a mobile gait analysis system which can accurately determine biomechanical data, including joint angles and ground reaction forces, in the lower limb amputee patient and also return clinically meaningful results to a prosthetist. Satisfying this need, would allow for better fitting prosthetic devices, which would in turn limit problems associated with and caused by an asymmetric gait.

### SUMMARY

Various system embodiments measure the full lower limb kinetics and kinematics of subjects outside of a motion capture gait laboratory. The system can determine the torques and forces at any of the joints in the body or in any of the limb segments. The system can be used for rehabilitation of patients experiencing injury to the lower extremity or amputee subjects. The system can also be used in the analysis and improvement of amputee prosthetics or joint replacement components. This system can also be used for the optimization of biomechanics in an athletic environment. Various embodiments can be employed a variety of commercial applications, including but not limited to rehabilitation, prosthesis evaluation and design, athletic performance, video game development and/or control.

Various embodiments satisfy the aforementioned need for a mobile gait analysis system which can accurately determine biomechanical data, including joint angles and ground reaction forces, in the lower limb amputee patient and also return clinically meaningful results to a prosthetist. Various embodiments, therefore, allow for better fitting prosthetic devices, and limit problems associated with and caused by an asymmetric gait.

In one embodiment, a method of performing gait analysis of a subject is provided. The method includes obtaining a plurality of measurement sets for a subject, each of the plurality of measurement sets including inertial measurements obtained from a sensor device associated with a different one of a plurality of segments of the subject. The segments can include a trunk or torso of the subject as well as limb segments. The method also includes calculating a sensor orientation for the sensor device associated with each of the plurality of segments based at least on a portion of a corresponding one the plurality of measurement sets and computing a segment orientation for each of the plurality of segments based on a data fusion process applied to each of the plurality of segments. The data fusion process includes combining at least a one of the plurality measurement sets and the corresponding sensor orientation to estimate the segment orientation. The method also includes determining joint angles based on the estimate of the segment orientation for each of the plurality of segments. Optionally, ground reaction forces can be obtained based force measurements.

In a second embodiment of the invention, there is provided a system for performing gait analysis. The system includes a processor and a communications interface configured for receiving a plurality of measurement sets, each of the plurality of measurement sets including inertial measurements obtained from a sensor device associated with different one of a plurality of segments of a subject. The system also includes a computer-readable medium having stored thereon a plurality of instructions for causing the processor to perform steps. The steps include calculating a sensor orientation for the sensor device associated with each of the plurality of segments based at least on a portion of a corresponding one the plurality of measurement sets and computing an estimate of a segment orientation for each of the plurality of segments based on a data fusion process applied to each of the plurality of segments, the data fusion process including combining at least a one of the plurality measurement sets and the corresponding sensor orientation to estimate the corresponding segment orientation. The steps also include determining joint angles based on the estimate of the segment orientation for each of the plurality of segments. Optionally, the steps can include computing ground reaction forces can be obtained based force measurements.

In a third embodiment of the invention, there is provided a sensor for analyzing gait and ground reaction forces. The sensor includes a forefoot portion removably attachable to a sole of a subject's forefoot and including at least one force sensor. The sensor also includes a heel portion removably attachable to a sole of the subject's heel and including at least one force sensor. The sensor further includes a processing unit communicatively coupled to the forefoot portion and the heel portion and configured for transmitting sensor signals from the forefoot portion and the heel portion to a remote computing device. In the sensor, at least one of the forefoot portion, the heel portion, and the processing unit includes at least one inertial measurement sensor. The sensor can be utilized with the systems and methods described herein. This embodiment also incorporates a method for calculating the biomechanical forces or moments at any location (prosthetic limb, tibial, or femoral locations for example) or any joint (prosthetic socket, knee, ankle, hip for example) in the biomechanic system for ambulation.

### BRIEF DESCRIPTION OF THE DRAWINGS

These and other features, aspects, and advantages of the present invention will become better understood with reference to the following description and appended claims, and accompanying drawings where:

FIG. 1 shows a system in accordance with the various embodiments for improving gait in a lower limb amputee fitted with a lower limb prosthesis;

FIG. 2 shows a block diagram of a configuration for IMU 106 in accordance with an embodiment of the invention;

FIG. 3 shows an exemplary configuration for the internal circuitry of an IMU in accordance with an embodiment of the present invention;

FIG. 4 shows an exemplary configuration for an IMU including an enclosure can be provided to house the circuitry of IMU;

FIG. 5A shows a top isometric view of forefoot portion in accordance with an embodiment of the present invention;

FIG. 5B shows a side view of a forefoot portion in accordance with an embodiment of the present invention;

FIG. 5C shows a top, disassembled view of forefoot portion in accordance with an embodiment of the present invention;

FIGS. 6A, 6B, and 6C illustrate exemplary embodiments of a heel portion of a footpad in accordance with an embodiment of the present invention;

FIG. 7 shows a footpad in accordance with an embodiment of the present invention;

FIG. 8 illustrates the exemplary configuration for footpad 114 of FIG. 7, that includes a forefoot portion and a heel portion, strapped to an athletic shoe, via one or more straps;

FIG. 9 shows an FPU coupled to footpad for purposes of providing power and data signals in accordance with an embodiment of the present invention;

FIG. 10, there is shown an exemplary configuration of footpad, that includes a forefoot portion and heel portion 60 in accordance with an embodiment of the present invention;

FIGS. 11 and 12 show operation of IMUs in accordance with an embodiment of the present invention;

FIGS. 13A and 13B schematically illustrate the operation of an algorithm in accordance with an embodiment of the invention.

FIG. 14 shows a schematic illustration of a method for utilizing sensor information for IMUs 106 to calculate knee angle in accordance with the various embodiments of the present invention;

FIG. 15 is a flowchart of steps in an exemplary method for adjusting a prosthesis in accordance with an embodiment of the invention;

FIG. 16 is a flow chart of steps in a method for evaluation of the sensors and the algorithms of the various embodiments of the present invention;

FIG. 17 illustrates an exemplary walking pattern evaluated with encoder data from a Mitsubishi PA-10 robot programmed to simulate walking motion using the sensors and the algorithms of the various embodiments of the present invention;

FIG. 18 shows the results of a sample trial of Walk A orientation angles from IMU data (MGAS values) and orientation angles calculated by the kinematic model (Robot values) for the “thigh” segment of the robot;

FIG. 19 shows the results of a sample trial of Walk A orientation angles from IMU data (MGAS values) and orientation angles calculated by the kinematic model (Robot values) for the “shank” segment of the robot;

FIG. 20 shows the results of a sample trial of Walk A calculated knee flexion angles (MGAS values) and knee flexion angles calculated by the kinematic model (Robot values);

FIG. 21 shows a sample trial of Walk A knee flexion angles from IMU data (MGAS values) and knee flexion angles from the kinematic model (Robot values) from one simulated gait cycle;

FIG. 22 shows a sample trial of Walk B calculated knee flexion angles (MGAS values) and knee flexion angles calculated by the kinematic model (Robot values);

FIG. 23 shows a sample trial of Walk B knee flexion angles from IMU data (MGAS values) and knee flexion angles from the kinematic model (Robot values);

FIG. 24 shows a sample trial of Stair Climb calculated knee flexion angles (MGAS values) and knee flexion angles calculated by the kinematic model (Robot values);

FIG. 25 shows a sample trial of Stair Climb knee flexion angles from IMU data (MGAS values) and knee flexion angles from the kinematic model (Robot values) from one simulated gait cycle;

FIG. 26 shows the flexion error from the different robot motions;

FIG. 27 shows plots of angle in three physiological planes for a thigh for conventional measurements and measurements in accordance with the various embodiments;

FIG. 28 shows plots of angle in three physiological planes for a shank for conventional measurements and measurements in accordance with the various embodiments;

FIG. 29 shows plots of angle in three physiological planes for a knee for conventional measurements and measurements in accordance with the various embodiments;

FIG. 30 shows plots of angle in three physiological planes for a heel for conventional measurements and measurements in accordance with the various embodiments;

FIG. 31 shows plots of angle in three physiological planes for a toe for conventional measurements and measurements in accordance with the various embodiments;

FIG. 32 shows plots of ground reaction forces for conventional measurements and measurements in accordance with the various embodiments;

FIG. 33 shows an exemplary computing device for carrying out the various embodiments.

Some of the figures illustrate diagrams of the functional blocks of various embodiments. The functional blocks are not necessarily indicative of the division between hardware circuitry. Thus, for example, one or more of the functional blocks (e.g., processors or memories) may be implemented in a single piece of hardware (e.g., a general purpose signal processor or a block or random access memory, hard disk or the like). Similarly, the programs may be standalone programs, may be incorporated as subroutines in an operating system, may be functions in an installed imagining software package, and the like.

It should be understood that the various embodiments are not limited to the arrangements and instrumentality shown in the drawings.

### DETAILED DESCRIPTION

The present invention may be understood more readily by reference to the following detailed description of preferred embodiments of the invention as well as to the examples included therein. All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

As noted above, a need exists for a mobile gait analysis system which can accurately determine biomechanical data, including joint angles and ground reaction forces, in the lower limb amputee patient and also return clinically meaningful results to a prosthetist. In view of the limitations of conventional methods, a novel mobile gait analysis system (MGAS) is provided, which can accurately determine biomechanical data, including joint angles and ground reaction forces, in the lower limb amputee patient. Further, the new MGAS can be used to return clinically meaningful results to the prosthetist, who can then determine how to adjust or modify a prosthetic lower limb device.

In particular, the various embodiments are directed to systems and methods for mobile gait analysis, force balancing, and alignment system to determine limb segment positioning, forces, and moments. The systems and methods are not limited solely to analysis of an affected limb, but to analysis of the intact limb. In the various embodiments, in order to mathematically determine joint moments and forces for purposes of adjusting and fitting a prosthesis, knowledge of several kinematic components is required. A first is knowledge of the ground reaction forces. The second is knowledge of limb orientation. Finally, knowledge of velocity and acceleration for the limb components, both linear and angular, is also required. The mobile gait analysis system of the various embodiments such data to be collected.

Although the various embodiments will be described with respect to human subjects with transtibial prostheses, this is solely for illustrative purposes. Rather, the systems and methods described herein can be utilized with either human or non-human subject having transfemoral or transtibial lower limb prostheses. Further, the systems and methods described herein are also not limited solely to use with prosthetic devices. Rather, the systems and methods described herein can also be used with healthy subjects, subjects with orthotic or other rehabilitative devices and conditions, and robotic devices and systems. The system may also be used to assist subjects with neurological or neurodegenerative conditions such as Parkinson's Disease, Multiple Sclerosis, Cerebral Palsy or other condition. This system may be used to measure or improve performance of non-human subjects such as race horses, dogs, or other animals.

In the various embodiments, the mobile gait analysis system allows quality kinetic and kinematic data to be measured without the infrastructure investment of a camera-based motion capture gait analysis facility. Moreover, as a laboratory setting is not needed, the mobile gait analysis system allows measurement of biomechanics data in any environment. That is, it can be utilized in the actual locations and activities that any subject or clinician desires to record or analyze biomechanics data. This allows rehabilitation or examination of the actual activities of daily living (ADL) subjects expect to regularly encounter. Further, a software system can be provided to assist with data interpretation. This software system will help assess component fit and alignment as well as patient biomechanics. However, analyses can also be performed manually in the various embodiments.

Beyond the known alignment systems based on force plates, this system represents a new approach to prosthesis alignment, fitting, and patient rehabilitation and will allow subjects to experience more natural and efficient function from their prosthetic limbs and reduce secondary disabilities. This approach will enhance the maintenance and performance of long-term prosthesis and socket performance/fit by increasing the ease of measurement of prosthesis performance. Mobile gait analysis also represents an evidence-based approach to prosthesis fitting and will allow wider use of evidence-based rehabilitation techniques. More natural function will allow patients to return to a higher level of activity, and the reduction in secondary disabilities includes chronic lower back pain, hip and knee pain, as well as osteoarthritis of the knee(s), hips or lower back.

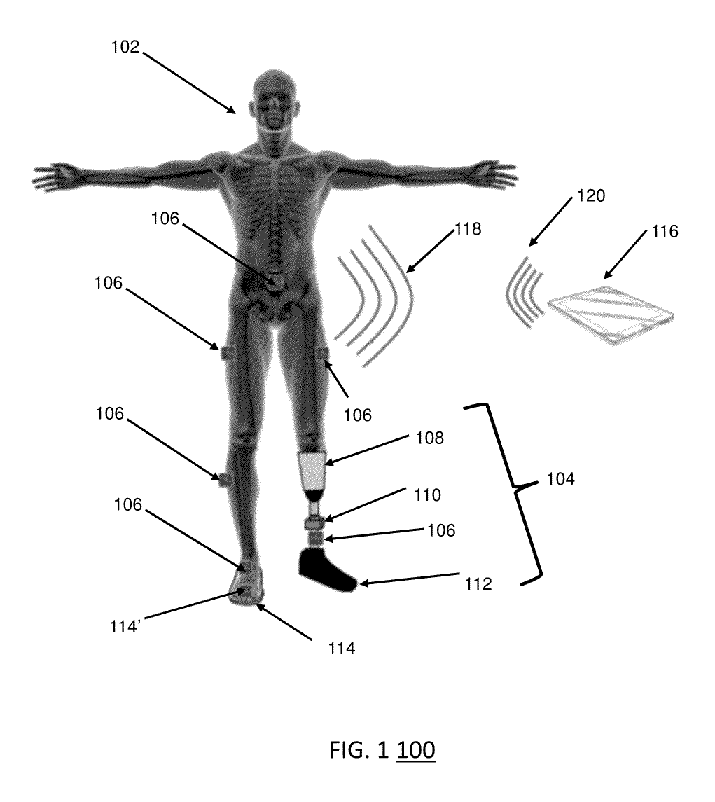

Turing first to FIG. 1, there is shown a system 100 in accordance with the various embodiments for improving gait in a lower limb amputee 102 fitted with a lower limb prosthesis 104. As shown in FIG. 1, an amputee 102 can be fitted with a plurality of inertial measurement units (IMUs) 106 on a torso segment (i.e., trunk segment) and various limb segments of the amputee 102. The IMUs 106 are configured to provide and communicate measurements of velocity (linear and/or angular), acceleration (linear and/or angular), orientation, gravitational forces, or any combinations thereof, at their respective locations using accelerometers, gyroscopes, magnetometers, geolocation devices (e.g., GPS), altimeters, or any combinations thereof. Thus, each IMU can consist of a single electronic component integrating features for conducting all necessary measurements in some embodiments. However, in other embodiments the IMU can be assembled from a collection of discrete devices. In some embodiments, the IMUs can be based on microelectromechanical system (MEMS) devices. However, the various embodiments are not limited in this regard and the IMUs 101 can be configured to utilize any other type of devices or combination of devices to provide the necessary elements.

As noted above, the amputee 100 is fitted with a prosthesis 104. In the exemplary embodiment of FIG. 1, the prosthesis 104 includes a socket 108 for interfacing with the amputee 102, one or more adapters 110, one or more IMUs 106, and a prosthetic foot 112. The prosthetic foot 112 can be substantially resilient or flexible in the various embodiments.

In some embodiments, the foot 112 can be connected to the remainder of the prosthesis 104 via a passive or powered ankle joint. Further, although the various embodiments will be discussed primarily with respect to below-the-knee amputees, the various embodiments are not limited in this regard and are equally applicable to above-the-knee amputees. Thus, in some embodiments, a passive or powered knee joint can also be included between the socket 108 and the remainder of prosthesis 104. Thus, prosthesis 104 can include a knee joint, an ankle joint, or both, each of which can be powered or passive.

The system 100 can also include a footpad 114 that is secured to the amputee's 102 healthy limb. The footpad 114 can comprise a plurality of force/moment (F/M) sensors. That is, sensors that measure loads and torques in one or more axes. For example, the footpad 114 can have an F/M sensor on a forefoot portion and can have an F/M sensor on a heel portion thereof. The F/M sensors can measure ground reaction forces (GRS) for the healthy limb. The F/M sensors can also determine the orientation of the foot or other portions of the healthy limb. In some embodiments, the footpad can include components similar to IMUs 106 to communicate measurements. In other embodiments, the footpad 114 can operate in conjunction with a foot processor unit 115, where the foot processor unit (FPU) 115 is configured to communicate with the footpad 114 and communicate measurements on behalf with footpad 114.

In some embodiments, the prosthetic foot 112 can be configured to operate in a manner substantially similar to footpad 114. Further, the IMU 106 in prosthetic leg 104 can also provide a FPU for the sensors in the prosthetic foot. However, in other embodiments, the prosthetic foot 112 can also be fitted with a footpad and, optionally, a FPU to collect measurements. The footpad 114 and IMU 106 may be integrated into a prosthetic foot with force and orientation measurement or feedback. The footpad may also be integrated into a shoe that can provide propulsive force and torque quantity and direction information. The data from the IMU is combined with the data from the footpad such that propulsive forces and torques, foot orientation, and propulsive force vector directions, with respect to a selected reference frame, are measured.

System 100 also includes a computing device 116 for communicating with various elements of system 100, such as IMUs 106, footpad 114, FPU 115, or any other components for providing sensor information. The communications links between computing device 116 and the various elements of system 100 can be wireless, wired, or a combination of both.

In operation, data from the IMUs 106 or from the footpad 114 can be transmitted as a signal 118 to computing device 116. The computing device 116 can also send a signal 120 to the IMUs 106 and/or footpad 114 (or FPU 115). In the exemplary configuration of FIG. 1, the signals 118 and 120 are transmitted wirelessly. However, the various embodiments are not limited in this regard and such data can be exchanged via wired connections or a combination of wireless and wired connections, as noted above.

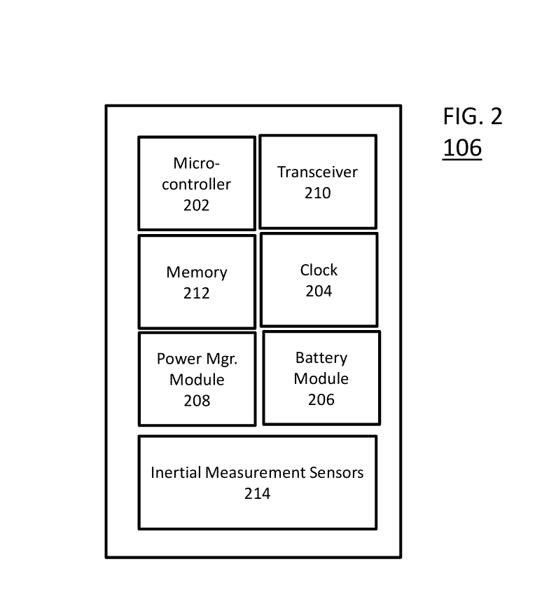

Referring to now to FIG. 2, a block diagram of a configuration for IMU 106 is shown. As illustrated in FIG. 2, an IMU can include a microcontroller 202 for controlling the operation of the IMU 106, a clock module 204, a battery module 206, a power management module 208, a transceiver 210, memory 212, and inertial measurement sensors 214.

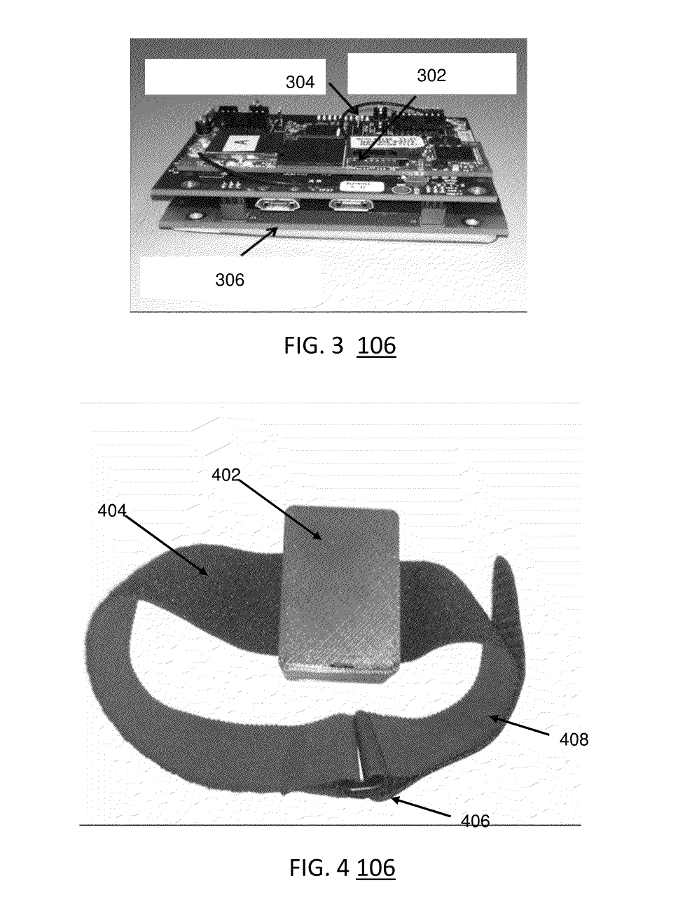

Referring now to FIG. 3, an exemplary configuration for the internal circuitry of an IMU 106 is shown. In particular, FIG. 2 shows the internal circuit of a microelectromechanical system (MEMS) IMU. In the exemplary configuration of FIG. 3, the IMU 106 is configured to include a computer-on-module 302, an expansion board 304, and a battery 306. The computer-on-module 302 can include an ARM processor running Minix and software written in C++, or any operating system and any suitable language. Further, this exemplary computer-on-module 302 can include Wi-Fi connectivity, Bluetooth connectivity, and/or microSD. However, the various embodiments are not limited in this regard and any other type of connectivity can be supported in the various embodiments. The expansion board 304 can include battery management circuits, an MSP430 microcontroller, a 3 axis IMU, a USB port, and/or a connection to a foot sensor. However, the various embodiments are not limited solely to the architecture described above and an IMU 106 can be configured with comparable functionality in a variety of other ways.

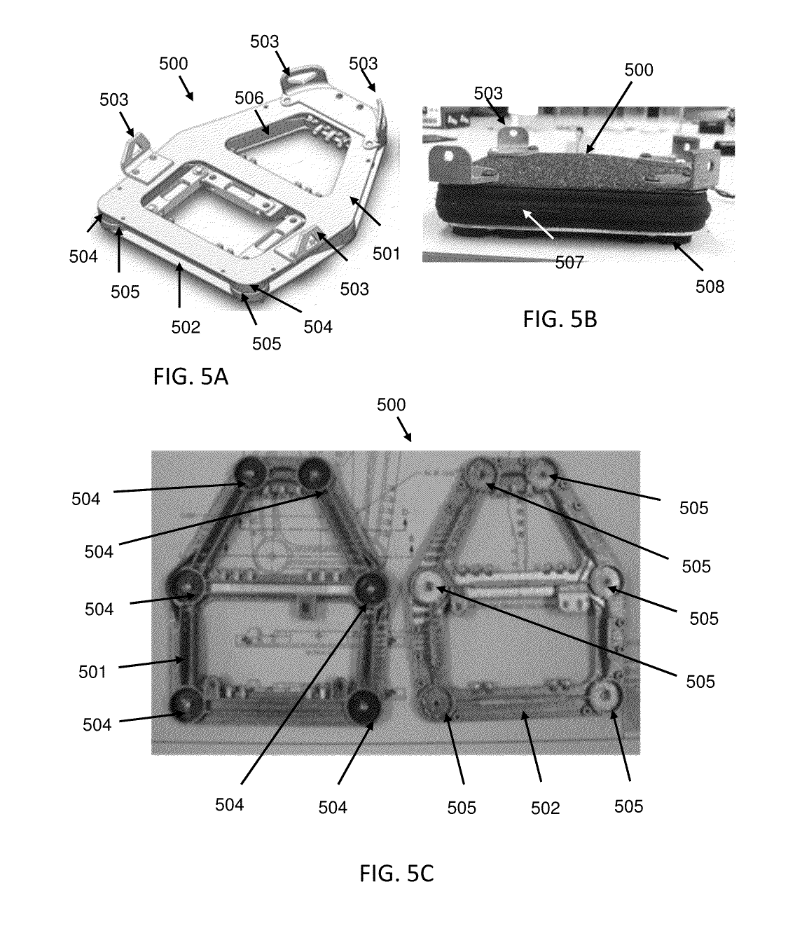

To provide IMUs 106 for the torso and limb segments, the IMUs 106 can be configured to allow their attachment to the body of amputee 102. For example, the IMUs 106 can be configured to be strapped to a plurality of limb segments at various points on an amputee's body. Referring to FIG. 4, an exemplary configuration for an IMU 106 is shown including an enclosure 402 can be provided to house the circuitry of IMU 106. The IMU 106 can be provided with a strap 404 as shown in FIG. 5. In this exemplary embodiment, the strap 404 can include adjustment rings 406 and hook and loop fastening portions 408. However, the various embodiments are not limited in this regard and other configurations for an IMU 106 can be provided to allow the IMUs 106 to be secured to an amputee 102 at various locations as shown in FIG. 1. For example, belts, clips, pins, and other methods of attachment are equally suitable.

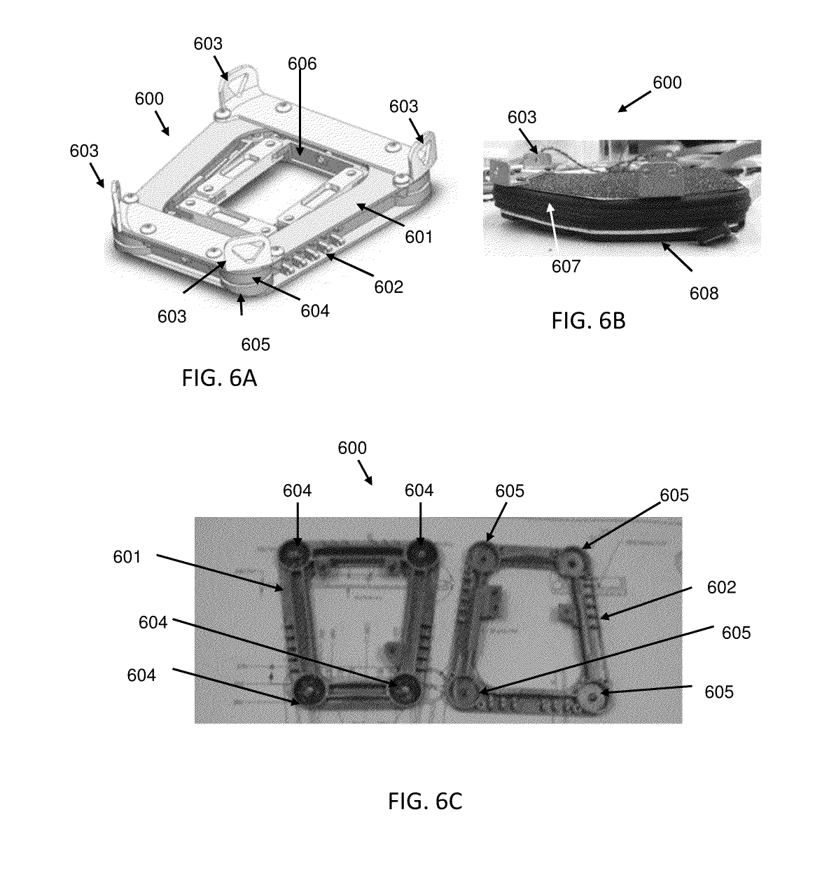

Referring now to FIGS. 5A-5C, 6A-C, and 7, one exemplary configuration for a footpad 114 is illustrated. In particular, FIGS. 5A-5C show an embodiment of a forefoot portion 500 of a footpad 114 (as illustrated in FIG. 1). FIG. 5A shows a top isometric view of forefoot portion 500. FIG. 5B shows a side view of forefoot portion 500. FIG. 5C shows a top, disassembled view of forefoot portion 500. As shown in FIGS. 5A-5C, the forefoot portion 500 can include a top plate 501 and a bottom plate 502. One or more cleats 503 can be secured to the top plate 501. The cleats 503 can serve to align the forefoot portion 500 with a wearer's foot or shoe. The top plate 501 can include one or more top sensor enclosures 504. The bottom plate 502 can include one or more bottom sensor enclosures 505. The top plate 501 can be secured to the bottom plate 502 by one or more fasteners (not shown). In the various embodiments, fasteners can include screws, nuts and bolts, clips, straps, adhesives, or any other device, material, or combination thereof for securing the top plate 501 to the bottom plate 502. The forefoot portion 500 is assembled by aligning and connecting one or more top sensor enclosures 504 to one or more bottom sensor enclosures 505 and securing via any fasteners. Additional structural support 506 can be provided to connect top plate 501 to bottom plate 502, such as a washer, spacer, or the like. As shown in FIG. 5B, the forefoot portion 500 of the footpad 105 can be enclosed in a flexible material 507, which can prevent debris, water, or contamination from entering the electronics or mechanical systems of the footpad 500. A tread 508 can be secured to the lower portion of the bottom plate 502 that contacts the ground, such that the forefoot portion 500 closely mirrors the sole of the prosthetic foot or the sole of a shoe placed thereon. The forefoot portion 500 can include 6 DOF Force Sensors (3 Loads, 3 Moments or Torques) to measure ground reaction forces.

Referring now to FIGS. 6A, 6B, and 6C, these illustrate embodiments of a heel portion 600 of a footpad 114 (as illustrated in FIG. 1). The structure of the heel portion 600 can be similar to that of forefoot portion 500. That is, the heel portion 600 can include a top plate 601 and a bottom plate 602. Further, one or more cleats 603 can be secured to the top plate 601. The cleats 603 can serve to align the heel portion 600 with a wearer's foot. Additionally, the top plate 601 can also include one or more top sensor enclosures 604 and the bottom plate 602 can include one or more bottom sensor enclosures 605. As in forefoot portion 500, the top plate 601 can be secured to the bottom plate 602 by aligning and connecting one or more top sensor enclosures 604 to one or more bottom sensor enclosures 605. Fasteners (not shown) can then be used to secure the plates 601 and 602 together. Additional structural support 606 can be provided to connect top plate 601 to bottom plate 602. As shown in FIG. 6B, the heel portion 600 of the footpad 105 can be enclosed in a flexible material 607, prevent debris, water, or contamination from entering the electronics or mechanical systems of the footpad 500. A tread 608 can be secured to the bottom of the bottom plate 602, as in forefoot portion 500.

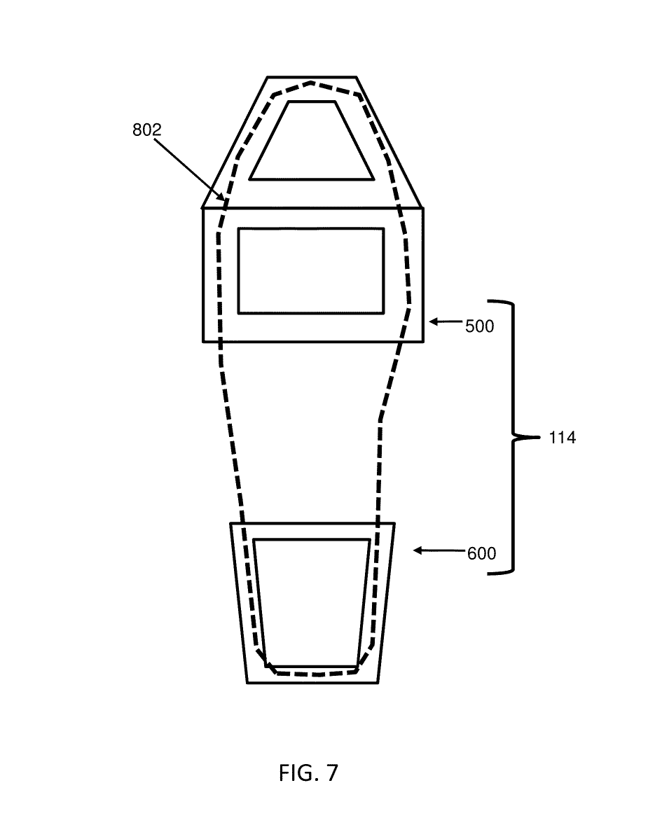

Together, forefoot portion 500 and heel portion 600 can be utilized to define the footpad 114 for foot 602, as shown in FIG. 7. Although FIG. 7 shows that the forefoot portion 500 and the heel portion 600 are physically separated, the various embodiments are not limited in this regard. In some embodiments, the portions 500 and 600 can be physically connected or incorporated into a same sole attachable to a foot or shoe. Thus, the footpad 114 can be formed using one or more components.

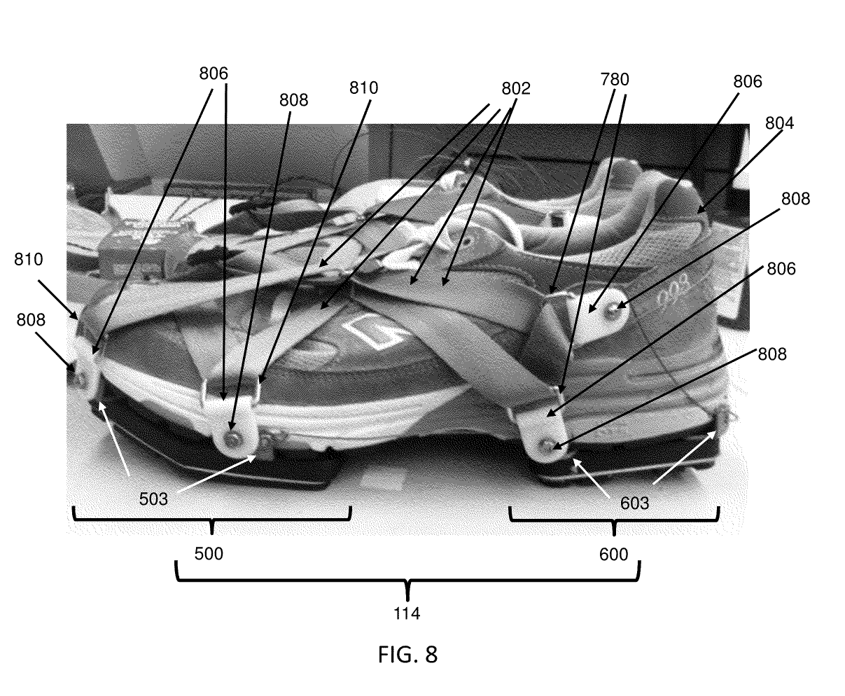

Now referring to now to FIG. 8, there is illustrated the exemplary configuration for footpad 114 of FIG. 7, that includes a forefoot portion 500 and a heel portion 600, strapped to an athletic shoe 804, via one or more straps 802. The straps 802 can be secured to the cleats 503, 603 of the forefoot portion 500 and the heel portion 600 via strap anchors 806. Each of the strap anchors 806 can comprise a fastener 808 for attaching a strap anchor 806 to one of cleats 503, 603. Further, each of strap anchors can also include a ring member 803 through which the strap 801 can be fed. The straps 802 can be arranged so that a friction fit keeps the footpad 114 attached to shoe 804. Alternatively, the straps 806 can be further arranged using one or more fasteners (not shown) to secure the straps 806 in place. For example, laces, hook and eye fasteners, fabric (elastic and non-elastic), cords (elastic and non-elastic), snaps, or buckles, screws, bolts, clip mechanisms or lugs to name a few. However, any other types of devices or methods useful for straps, belts, string, or rope can be used in the various embodiments.

Although a specific arrangement of straps and anchors is illustrated in FIG. 8, the configuration can vary in the various embodiments. Further, other types of fasteners can be utilized in the place of straps 801. For example, clips, clamps, belts, or any other fastening devices can be used in the various embodiments. Additionally, the footpad 114 can be incorporated directly into shoe 804 or screwed, bolted, or clipped in some embodiments.

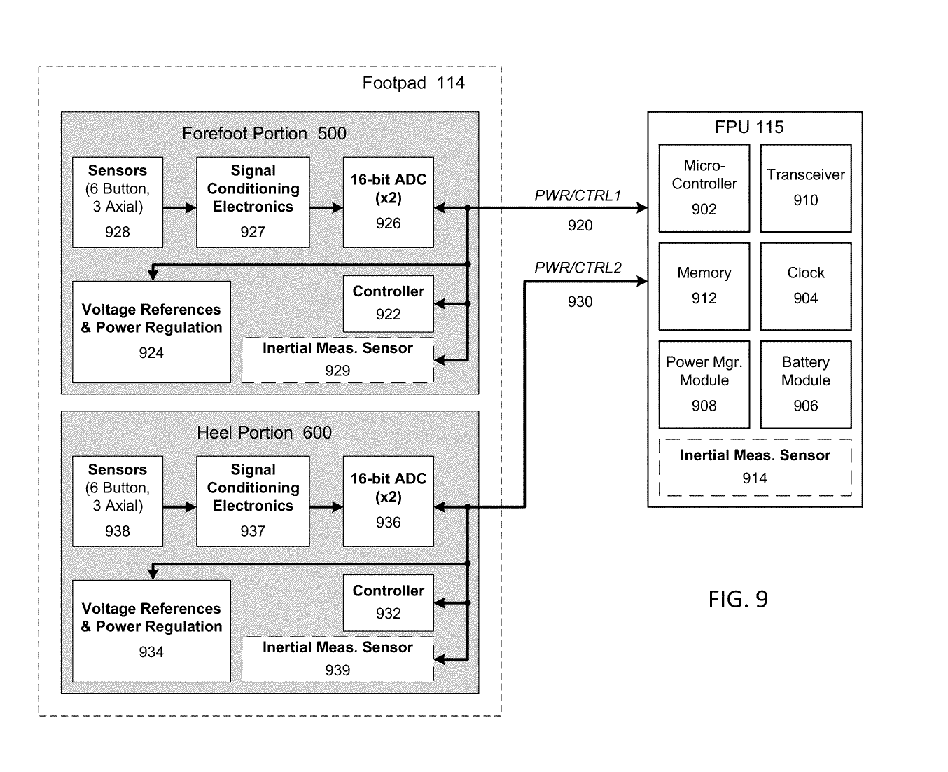

As noted above, the footpad 114 can be configured in a manner similar to IMU 106 or can include a FPU 115 that operates with footpad 114 and that can also be removably attached to the foot (healthy or prosthetic) of the subject. Regardless of the configuration, the block diagram of FIG. 9 illustrates an exemplary configuration for the circuitry of the footpad 114 or footpad 114 with FPU 115. For ease of illustration, FIG. 9 will be discussed in terms of a footpad 114 and an FPU 115.

Referring to FIG. 9, there is shown an FPU 115 coupled to footpad 114 for purposes of providing power and data signals. Specifically, FPU 115 is coupled to forefoot portion 500 via a first link or connection 920 and FPU 115 is coupled to heel portion 600 via a second link or connection 930. These connections 902, 904 can each include one or discrete links for providing data and/or power. FPU 115 can be configured in substantially a same manner as IMU 106 in FIG. 2. That is FPU 115 can include a microcontroller 902, a clock 904, a battery module 906, a power management module 908, a transceiver 910, and a memory 912.

Some elements of each of forefoot portion 500 and heel portion 600 have already been described above. However, to allow operation with FPU 115, additional elements can be provided. For example, as show in FIG. 9, forefoot portion 500 includes a controller 922 for controlling operation in forefoot portion 500 and receiving instructions from FPU 115. The forefoot portion 500 also includes a power regulation module 924 for receiving power signals from the FPU 115 and providing appropriate power to other components of forefoot portion 500. To provide an output signal, the forefoot portion 500 includes signal conditioning electronics 927 and an analog to digital (A/D) converter 926 to convert the analog signals from the sensors 928 to digital signals for use by the FPU 115 and components beyond. A similar configuration can be provided for heel portion 600. Thus components 932, 934, 936, 937, and 938 in heel portion 600 operate in substantially a same manner as components 922, 924, 926, and 928 in forefoot portion 500.

It should be noted that in some embodiments, controllers 922 and 932 are not utilized. In such embodiments, the forefoot portion 500 and heel portion can be controlled via microcontroller 902. Additionally, the arrangement in FIG. 9 is for exemplary purposes only and in other embodiments, a different arrangement of the components can be used, with more or less components than shown in FIG. 9.

In the various embodiments, it can be advantageous to provide inertial measurement sensors at the foot. For example, for purposes of gait analysis, it is not only use useful to obtain the orientation of a foot (healthy or prosthetic), but also necessary, as direction and orientation are relatively significant for any type of gait analysis. Thus, in some embodiments the FPU 115 at the foot can be configured in substantially the same way as an IMU 106. That is, the FPU 115 can include inertial measurement sensors 914. However, in other embodiments, inertial measurement sensors, or any other type of sensor, can be provided within the footpad 114 as well. For example, inertial measurement sensors 929 and 939 can be configured for measuring orientation for at least one of the forefoot portion 500 and the heel portion 600, respectively.

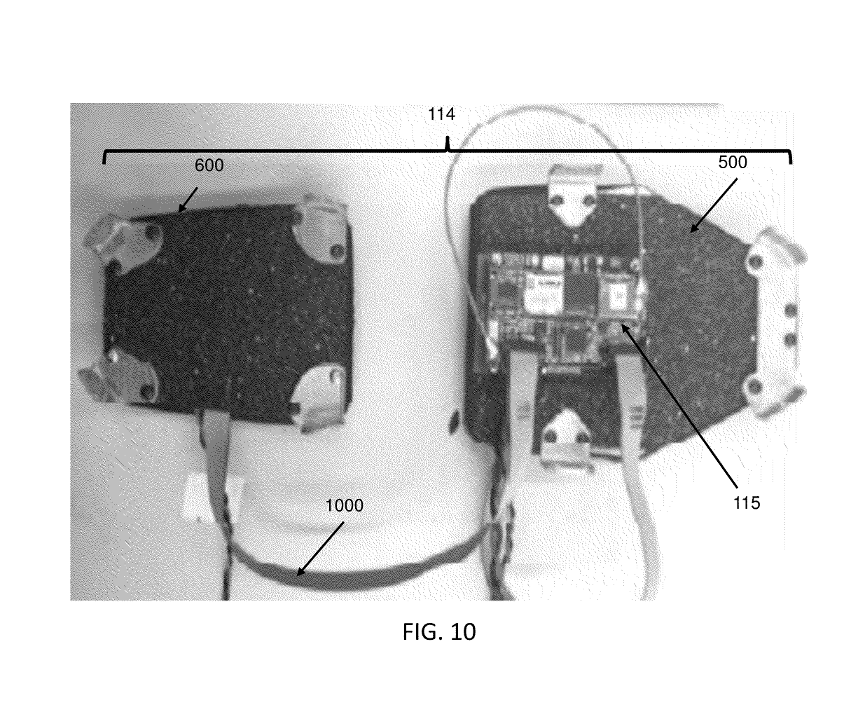

Now referring to FIG. 10, there is shown an exemplary configuration of footpad 114 that includes a forefoot portion 500 and heel portion 600, as described above. In the configuration of FIG. 10, there is shown the FPU 115 within forefoot portion 500. As noted above, the FPU 115 can be used to power and/or control one or more sensors, to store data from the one or more sensors, and/or two transceivers data from one or more sensors. A connector 1000 can be used connect the FPU 115 in forefoot portion 500 to the heel portion 600. The connector 1000 can include one or more wires to communicatively connect with the sensors and other devices in footpad 114. However, the configuration of FIG. 10 is provided solely for illustrative purposes. That is, FPU 115 can be coupled to a footpad 114 in a variety of ways, including using one or more wired connections, one or more wireless connections, or any combination thereof.

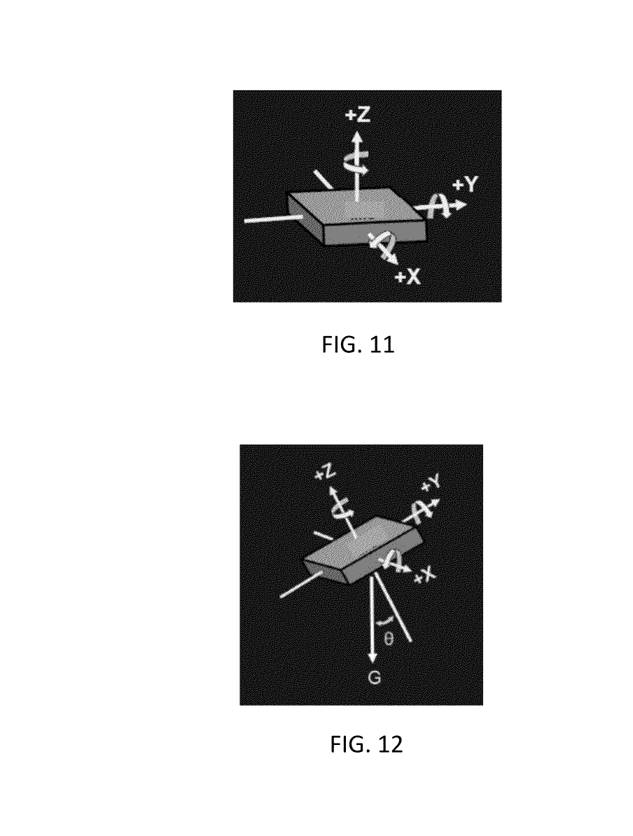

Now turning to FIGS. 11 and 12, the operation of IMUs 106 discussed above is described. Each of the IMUs 106 can include one or more three axis gyroscopes to measure angular rates of change and one or more 3 axis accelerometers to measure acceleration. As noted above, such an IMU can be fabricated using MEMS devices. In operation, the signals from the gyroscope can be integrated to calculate orientation of the IMU 106 in space. The force of gravity, as shown in FIG. 12B, can be used to determine the pitch and roll of each IMU 106. Thereafter, this information for each of IMUs 106 (each of which is associated with a limb segment or forefoot or heel footpad segment) can be individually processed using a data fusion technique to then provide information for each corresponding segment. In some embodiments, the data fusion techniques or processes can include Kalman Filtering processes, such as Extended Kalman Filtering (EKF). Such data processing allows for the extraction of better results than by relying solely on accelerometer or gyroscope measurements alone.

Although exemplary embodiments below will be described with respect to an algorithm based on an extended Kalman filter process, the various embodiments are not limited in this regard. Rather, in the various embodiments, any other type of data fusion techniques or processes can be utilized.

Generally, pitch and roll angles can be calculated by estimating the direction of gravity using the accelerometer signals. Alternatively, the gyroscope signals can be integrated to determine pitch, roll and yaw (heading). However, these calculations are subject to drift and noise which cause increasing error as the signal is integrated over time. Individually, these respective angle calculations are inaccurate. Accordingly, an extended Kalman filter process was developed in the various embodiments to fuse the accelerometer and gyroscope data to estimate segment orientation while accounting for noise and drift.

In one particular embodiment, the filter uses a 14-element state vector (1)

$$x = [ \begin{matrix}{v}_{int} \\ {a}_{int} \\ {ω}_{body} \\ {b}_{gyr} \\ r \\ p\end{matrix} ] \qquad ( 1 )$$

where vᵢₙₜ and aᵢₙₜ are velocity and acceleration in three axes transformed to an intermediate reference frame, ω_{body} and b_{gyr} are the gyroscope signals and the gyroscope bias in three axes, and r and p are roll and pitch of the segment. The intermediate reference frame can be initially aligned with a laboratory reference frame but rotates about the gravity vector and is propagated outside of the roll and pitch EKF. The rotations from the laboratory reference frame to the IMU frames can be represented using direction cosine matrices so pitch and roll rotations can be isolated while rotations about the gravity vector are ignored.

The orientation about the gravity vector (internal/external rotation, heading or yaw) can be calculated through a second EKF that assumes the yaw rotation is minimal and about 0 degrees. This is a valid assumption when the relative rotation between segments (joint rotations) is being examined as opposed to the absolute rotation in global space.

The velocity at step k+1, in the intermediate frame are found by numerically integrating aᵢₙₜ (2) over timestep Δt.

v_{int,k+1}=v_{int,k}+a_{int,k}Δt. (2)

Accelerations, angular rates and angular biases are modeled by using the value at the previous time step, adding noise, w^{a}, w^{ω}, w^{gyr} to acceleration, gyroscopes and bias and, for the acceleration model subtracting a factor multiplied by velocity, yvₚ, to stabilize the velocity calculation (3)

a_{int,k+1}=a_{int,k}+wₖ^{α}−yv_{p,k}

w_{body,k+1}=ω_{body,k}+wₖ^{α}

b_{gyr,k+1}=b_{gyr,k}+wₖ^{gyr} (3)

Angles of the segments in the laboratory reference frame can then be calculated by transforming the gyroscope signals to the laboratory reference frame (4)

\dot{r}=ωₓ+(ωx cos r+ω_{y} sin r)tan p

p=(ω_{y} cos r−ωₓ sin r)

\dot{y}=(ωₓ cos r+ω_{y} sin r)sec p (4)

representing the time derivative of roll, pitch and yaw, \dot{r}, \dot{p}, and ÿ, then numerically integrating the angular velocities (5)

rₖ₊₁=rₖ+\dot{r}ₖΔt

pₖ₊₁=pₖ+\dot{p}ₖΔt

yₖ₊₁=yₖ+\dot{y}ₖΔt (5)

Here, y is yaw, which is not included in the pitch and roll EKF state equations and represents the rotation of the intermediate reference frame about the gravity vector.

The measurement vector (6) consists of the three signals from the accelerometer, aᵢₘᵤ which include the gravity vector in the IMU frame, three signals from the gyroscope in the IMU frame and any drift associated with the gyroscope, w_{IMU} and b_{gyr}. An estimate of roll and pitch, rₑₛₜ and pₑₛₜ, respectively, is calculated using the direction of gravity from the accelerometer signals, basic trigonometry and sequential rotations. The filtered measurement vector, vₖ can then be defined by:

$${v}_{k} = ( \begin{matrix}{a}_{IMU , k} \\ {ω}_{IMU , k} + {b}_{gyr , k} \\ {r}_{ext} \\ {p}_{ext}\end{matrix} ) + {w}_{k}^{max} , \qquad ( 6 )$$

where wₖ is the measurement noise at time k.

The process covariances were calculated using the ideal signals calculated with a kinematic model. Only covariances for aᵢₙₜ, ω_{body}, and b_{gyr} were used. All other covariances were set to zero. The angle calculated using the gravity signal from the accelerometers is only accurate during low linear acceleration (when the IMU is not in motion). Therefore, the measurement covariances were optimized so that the algorithm weights the gyroscope measurements more heavily than the accelerometer and estimated angle measurements. The covariances may also be adjusted in real time based on the IMU data or by using data from the force sensors on the feet. During times of low linear acceleration the covariances can be adjusted so as to weight the accelerometer measurements and the pitch and yaw calculations from gravity more heavily than the gyroscope measurements.

In the various embodiments, the gravity vector can be obtained in a variety of methods. One method is to use accelerometers, such as those in the IMUs. Accelerometers use a “proof mass” which experience forces when the sensor is subjected to acceleration. This can cause a movement of the mass and a strain in the members that support the mass. Typically MEMS devices, like those described above, use capacitive components supported on flexures with integrated capacitive elements to measure the deflection of the proof mass. The changes in capacitance can be measured as a change in acceleration or a feedback voltage can be used to maintain the proof mass at a constant location. The amount of voltage required to hold the proof mass in place is proportional to the acceleration.

If a single axis accelerometer is stationary and oriented such that the measurement axis is perfectly vertical, the mass will deflect due to gravity and the accelerometer will detect an acceleration that equals 9.81 m/s² or 1 g. The three axis accelerometers used herein consist of three single axis accelerometers placed orthogonal to each other. Therefore, when the sensor is not moving or experiencing no inertial acceleration, the only signal the accelerometer senses is that of gravity deflecting the proof masses. If one of the three axes is perfectly aligned to vertical it will sense 1 g of acceleration and the other axes will read 0 g. If the accelerometer is randomly oriented and no single axis is aligned with gravity, then the proof masses in the other axes will also deflect and the gravity component in each of the axes will be detected. Assuming that the sensor is not moving, the orientation of the sensor can therefore be calculated using the known magnitude of gravity, the gravity component in each axes, and trigonometric functions arcsine and arccosine.

Depending on the amount of motion, the covariance values can be adjusted for the gyroscope signals and the accelerometer signals. That is, the angle values are either calculated primarily based on a direction of gravity calculated by the accelerometers or by using the gyroscope signals. During motion, an algorithm can be weighted to consider the gyroscope signals more and the angle calculated from gravity less, because using the accelerometers is less accurate when an IMU is in motion. When the IMU is not moving, the covariance values can be adjusted to consider the angle calculated from gravity using the accelerometers more, because that calculation should be of higher accuracy. This provides a correcting measurement which mitigates the effect of gyroscope drift over time.

As noted above data fusion methods, such as the EKF methods described above, provide only a portion of an overall algorithm in accordance with the various embodiments. For example, the overall algorithm provides parameters that are input to the data fusion portion and also determines when motion is occurring and when motion has ceased.

In operation, the parameters that are input to the data fusion portion of the overall algorithm can be divided into recursive-type and non-recursive-type parameters. The overall algorithm is configured to provide initial values for both types of parameters. New values for the recursive-type parameters are then calculated by the data fusion portion during a sampling/calculation period and fed back into the data fusion portion as an input for the next sampling/calculation period. The non-recursive-type parameters, such as the measurement covariance values, are also fed into the data fusion portion but remain unchanged by the data fusion portion. However, the overall algorithm may change these non-recursive-type parameter values over time.

The overall algorithm determines when motion starts by using a threshold value for the gyroscope signal of each respective IMU. Also, depending on what portion of the gait activity (stance phase or swing phase) the particular limb is experiencing, the overall algorithm can change the measurement covariance and can determine how the data fusion algorithm for each segment will weigh the various measurement signals. For covariances for the thigh, shank, and trunk segments, only the IMU data is used as a measure to adjust the measurement covariances. For the foot segments, the force measurements are used to determine when the foot is on the ground and in a stance phase. However, these force measurements are only used to adjust the measurement covariances outside of the data fusion segment and are not used in the orientation calculation.

Other parameters that the overall algorithm calculates for input to the data fusion segment include an initial gyroscope bias which is taken from an average of a static data collection from the respective IMUs. The standard deviations of the gyroscope signals from these static trials are also used as the basis for some of the measurement covariance values.

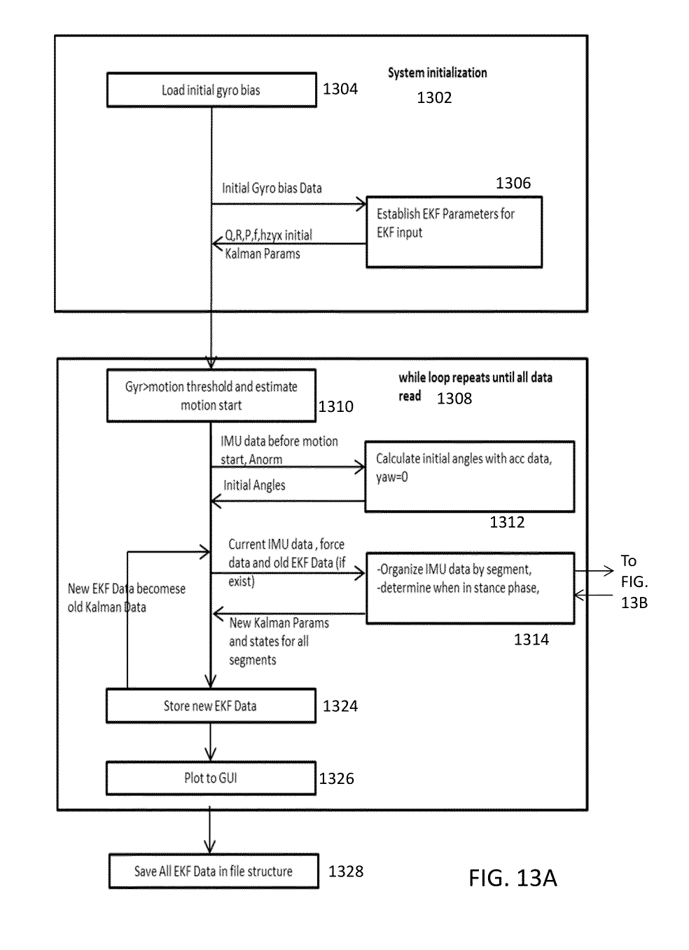

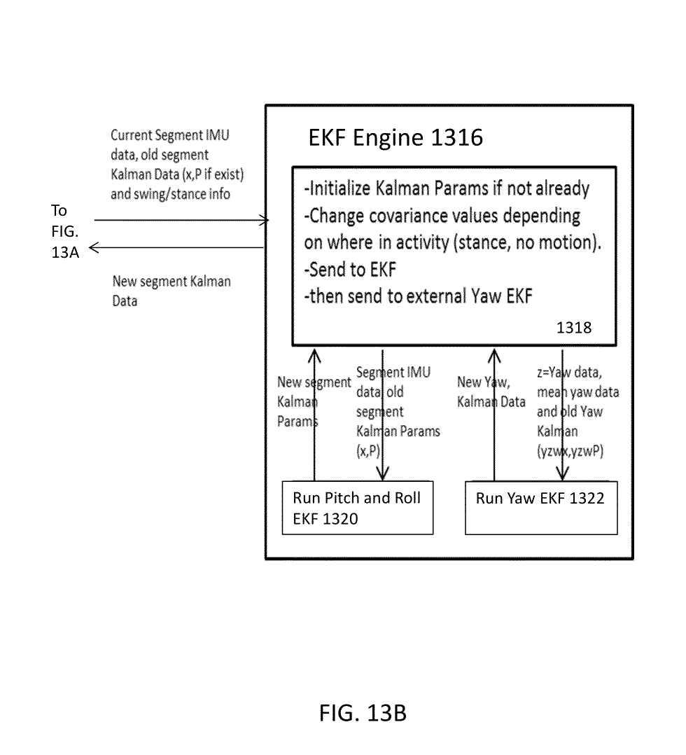

The operation of the overall algorithm is described below in greater detail with respect to FIGS. 13A and 13B. FIGS. 13A and 13B schematically illustrate the operation of the overall algorithm in accordance with an embodiment of the invention. The overall algorithm can begin with an initialization process 1302. The initialization process begins with a loading of the initial gyroscope bias at 1304. Based on the initial gyroscope bias, EKF parameters for inputting to the EKFs for each segment are generated. These include initial values for:
Q: Process noise covariance. In some embodiments, this can be set to 0.
R: Measurement noise covariance matrix. This is square matrix, such as an 8×8 matrix. The values along the diagonal of R are the covariances and determined using a combination of measurements (static gyroscopic standard measurements) and factors that are determined empirically.
f: State function which takes measurements and turns them into states.
hyxz: Measurement function which relates states back to the measurements using a yxz rotation sequence.
   Once the initial gyroscope bias and initial EKF parameters are obtained, these values are passed to a loop 1308 that is performed to generate EKF output data.

Loop 1308 first begins with a determination that motion has started. In particular, at 1310, a determination is made that motion was started if gyroscope measurements exceed a threshold value for motion start. If motion start is determined at 1310, initial angles for the segments are generated at 1312. In particular, IMU data before motion, specifically acceleration data (Aₙₒᵣₘ), is used at 1312 to calculate initial angles using yaw=0. The loop 1308 can then utilize EKFs to get data for each of the segments.

In particular, at 1314. the current IMU data, force data, and old EKF data (if available) is received. The IMU data can then be organized by segment at 1314. Further, a determination of whether a current phase is a stance or a swing phase can be made at 1314. Once the IMU data is organized and a current phase (swing or stance) is determined, the EKF process can be performed by an EKF engine 1316.

The EFK engine 1316 receives as inputs current IMU data, old or previous EKF data (if available), and the current phase information (i.e., swing or stance). The old or previous EKF data can include x, the 14 state vector of equation (1), and P, prior variance estimates for the states. Initially, P can be initialized as a square (14×14) matrix of zeroes. In the data fusion portion, the following is then performed at a configuration stage 1318:
(1) Any EKF parameters not yet initialized are initialized
(2) Covariance values are adjusted based on the current activity (i.e., swing or stance phase)
(3) Parameters are forwarded to EKF filters for processing.
   As noted above, two types of EKFs are run, a pitch and roll EFK 1320 and a yaw EKF 1322. For the pitch and roll EKF 1320, it is passed data that include the IMU data for the segment of interest and the current pitch/roll EKF parameters for the segment (x, P). In response, the EKF 1320 generates new values for these EKF parameters. For the yaw EKF 1322, it is passed current yaw data (z), mean yaw data, and old or current yaw EKF parameters for the segment. For example, x_{yaw} (yaw state) and P_{yaw}. (yaw variance estimate) In response, the EKF 1322 generates updated yaw data and updates the yaw EKF parameters.

Thereafter, the updated EKF parameters from EKFs 1320 and 1322 are passed back to loop 1308 at 1314 and the updated EKF parameters and states of the segments (i.e., the updated segment orientations) are then provided. The updated data can then be stored at 1324. Further, the updated data can be passed back into loop 1308 to provide the old or current EKF data for a next iteration performing 1314 and 1316. The stored data can then be plotted or otherwise presented to the user at 1326. Finally, all EKF data generated by loop can be stored at 1328.

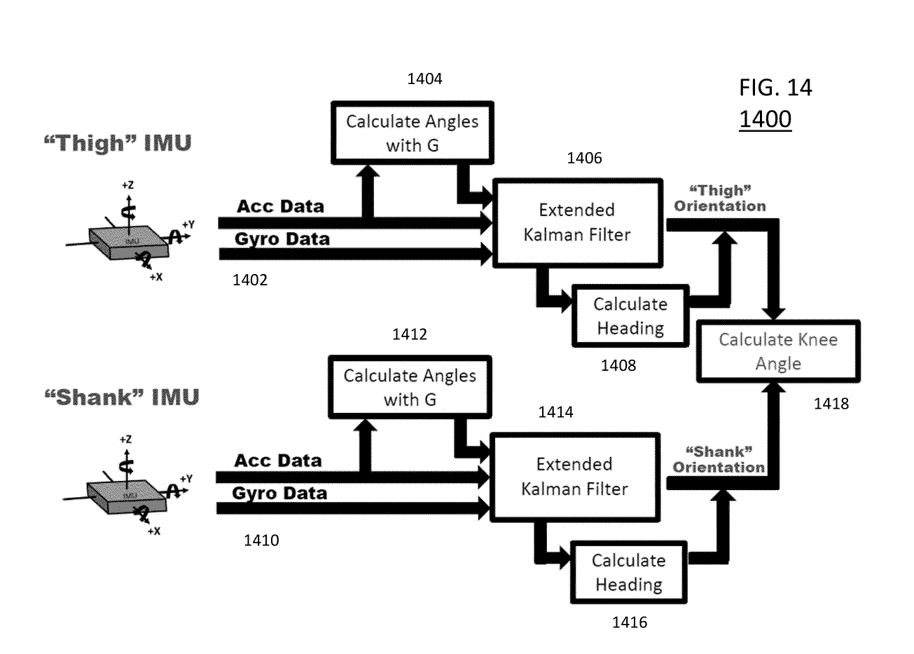

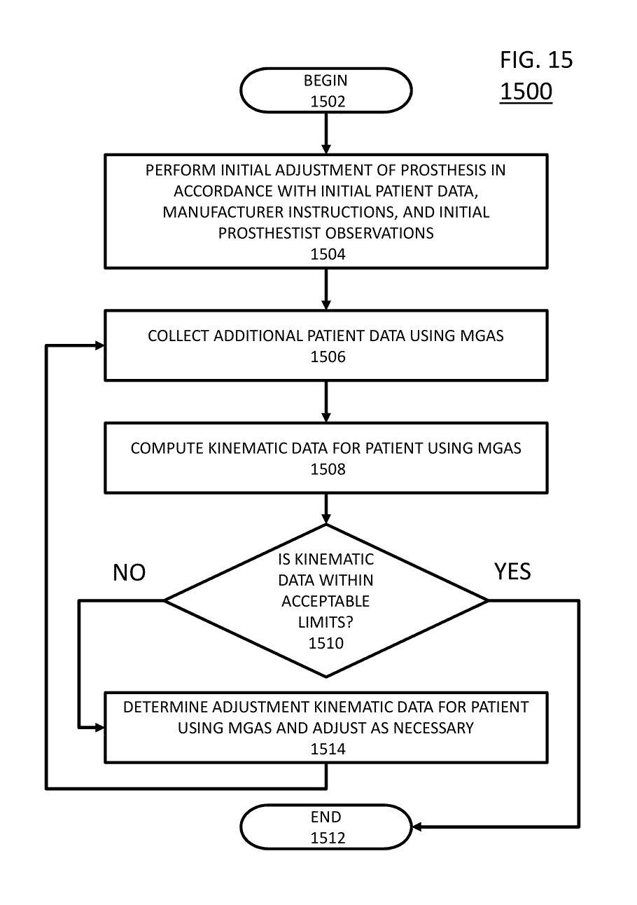

The filtering process described above can be used to carry out the methods of FIGS. 14 and 15. Turning first to FIG. 14, there is shown a schematic illustration of a method 1400 for utilizing sensor information for IMUs 106 to calculate knee angle in accordance with the various embodiments. The method begins at step 1402 in which an IMU 106 associated with a thigh, for which knee angle is to be calculated, generating and delivering accelerometer and gyroscope data. Thereafter at step 1404, the angle of x, y, and z axes are calculated with respect to a direction of gravity (G), as discussed above with respect to FIG. 12B. The angle information from step 1404, along with accelerometer and gyroscope data from step 1402 is then fed into the extended Kalman Filter at step 1406 to compute a current pitch and roll of the thigh. Additionally, at step 1408, the heading for the thigh can be calculated based on the heading angular speed obtained from the Kalman filter. Together, the heading (i.e., yaw), the pitch, and the roll therefore provide the orientation of the thigh. The processes at 1404-1408 can be carried out as described above with respect to FIGS. 13A and 13B.

The shank orientation can be computed in a substantially similar way. In particular, steps 1410, 1412, 1414, and 1416 can be performed for the data from an IMU 106 associated with a shank, for which the knee angle is to be calculated, in substantially a same way as steps 1402, 1404, 1406, and 1408, respectively. Steps 1410-1416 can be performed at a same or different time as steps 1402-1408. However, for purposes of reducing errors, it is preferable to perform these steps concurrently so that the calculated orientation is based on corresponding measurements of the thigh and shank. Once the orientation of each of the thigh and shank is obtained at steps 1406, 1408, 1414, and 1416, the associated knee angle can be calculated at step 1418. The method 1400 can then be repeated to capture development of the knee angle over time. Further, the method 1400 can be performed for both legs, concurrently. Additionally, a similar process can also be performed for the individual segments of the footpad (i.e., the forefoot and heel). Thus, in addition to capturing knee angle, angles for the ankle and angle associated with the foot can also be captured. In such configurations, the ground reaction forces can be considered.

As a result, the kinematics of the legs of the amputee can be captured without the need for a conventional gait laboratory setup. Therefore, together with other information regarding the prosthesis and the amputee and without the need of a gait laboratory, the prosthetist can perform adjustments to improve the gait of the amputee until such kinematics are within acceptable tolerance limits. For example, a method of carrying out such adjustments is presented below with respect to FIG. 15.

FIG. 15 is a flowchart of steps in an exemplary method 1500 for adjusting a prosthesis in accordance with an embodiment of the invention. The method 1500 begins at step 1502 and proceeds to step 1504. At step 1504, the amputee can be fitted with a prosthesis. At this step, the prosthesis can be initially adjusted based on patient data (e.g., height, weight, previous prosthesis configuration), manufacturer recommendations for configuring the prosthesis, and any initial observation of the prosthetist with regards to operation of the prosthesis or to adjust cosmetic issues with the prosthesis or the patient's gait.

Once the initial configuration of the prosthesis is completed, the method 1500 can proceed to step 1506 to collect additional patient data using the MGAS systems described herein. For example, the amputee/patient can be outfitted as described above with respect to FIG. 1. Thereafter, the patient can walk while data is collected from the various sensors. Once the patient data is collected at step 1506, the kinematic data can be computed at step 1508. This kinematic data can be computed as described above as described above with respect to FIGS. 13A, 13B, and 14.

The prosthetist, manually or programmatically, can then evaluate the kinematic data. In particular, at step 1510, the kinematic data for the patient can be evaluated to determine whether or not if falls within acceptable limits. For example, to provide a proper gait for the user, it may be preferred that the development of knee angles on both the healthy and affected legs be symmetric. Further, it may also be preferred that the overall stride of the healthy and affected legs also be similar as possible. Thus, a determination is made whether there are any unbalanced or irregular aspects of the patient's gait for the current prosthesis configuration.

If the kinematic data is within acceptable limits at step 1510, the method 1500 can end at step 1512. If the kinematic data is not within acceptable limits at step 1510, the method 1500 can proceed to step 1514. At step 1514, further adjustments can be made to the prosthesis, based on the kinematic data and other data. Such adjustments can be determined manually or automatically based on the kinematic and other data. Thereafter, the method can repeat steps 1506, 1508, 1510, and 1514 until the kinematic data is within acceptable limits.

Although the various embodiments have been described with respect to evaluating gait with respect to the development of the knee angle over time, the various embodiments are not limited in this regard. Substantially similar methods can be utilized for purposes of evaluating the kinematics of any of the other joints of the amputee and/or the prosthesis. For example, similar methods can be applied to adjust the gait of below-the-knee amputees by using measurements of ankle angle to determine how to adjust the prosthesis. In another example, similar methods can be applied to adjust a prosthesis including both knee joints and ankle joints.

### EXAMPLES

Although examples of various aspects of the present invention are discussed in detail below, the various embodiments are not limited in this regard. Rather, these examples are provided solely for illustrating or clarifying the various embodiments of the present invention.

In the following examples, the method of extended Kalman filtering of the various embodiments was evaluated conducting experiments to confirm whether the data collected by the IMUs and the kinematic data obtained using the extending Kalman filtering corresponded to actual kinematic data. In particular, these experiments employed using a Mitsubishi Heavy Industry (Tokyo, Japan) PA-107C robot arm with IMUs and a data processing system, as described above. The difference between applying the EKF algorithm to data from a robot and data from a human subject is adjusting the measurement covariance values, or adjusting how much the EKF weighs one signal over another. The robot was controlled with a personal computer which moved the arm through a repeatable motion sequence while simultaneously recording the encoder data from the robot. A separate personal computer recorded the gyroscope and accelerometer data from the IMUs.

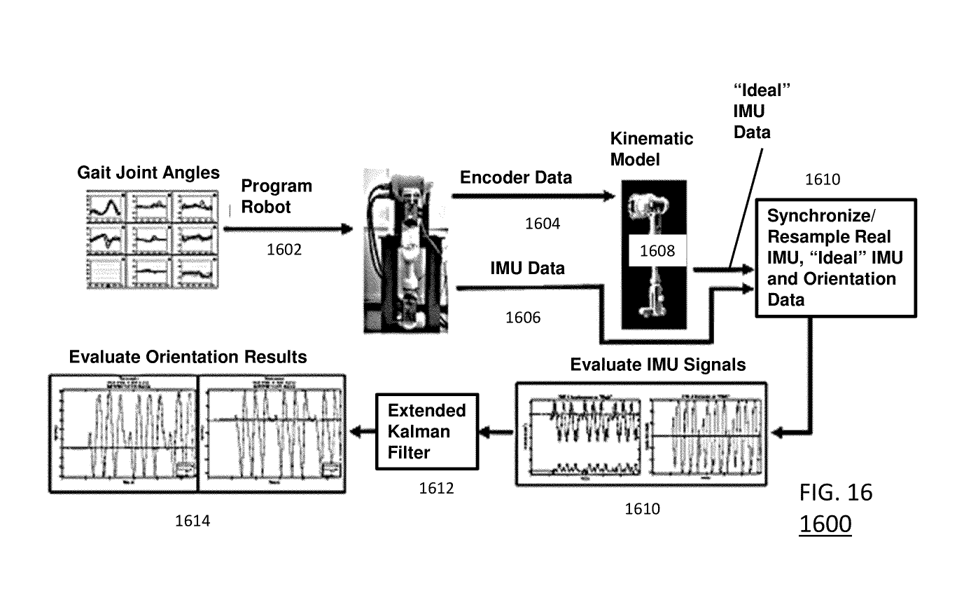

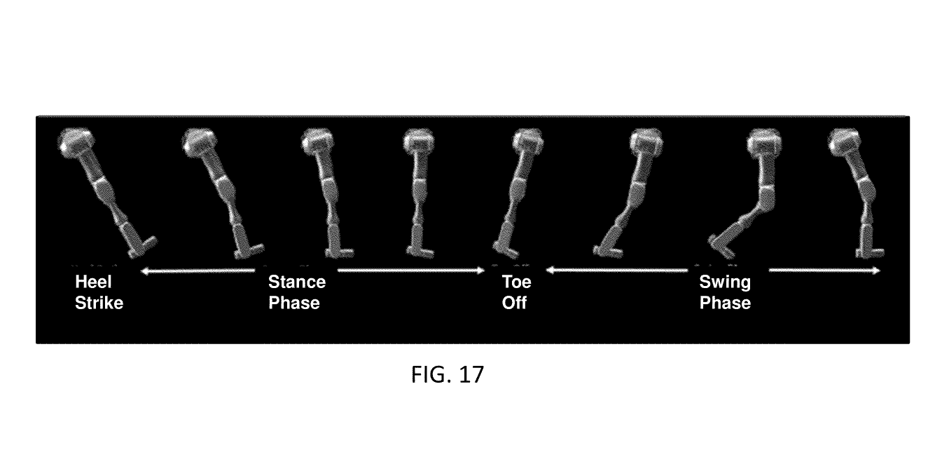

The overall process for the experiment is shown in FIG. 16. FIG. 16 is a flow chart of steps in a method 1600 for evaluation of the sensors and the algorithms of the various embodiments. First, at step 1602, the robot was programmed and actuated to simulate the motion of a human leg using joint angle data from gait analyses of a healthy subject. The human leg has nine rotational degrees of freedom (DOF) including the hip, knee and ankle while the robot can only represent six, thus, three DOF are excluded from the robot motion. The angles represented by the robot included hip flexion/extension, hip abduction/adduction, knee flexion/extension, abduction/adduction and internal/external rotation and foot flexion/extension. The motions evaluated for these experiments consist of two different walking patterns, Walking A (illustrated in FIG. 17) and Walking B. Walking B is slightly faster than Walking A, but simulates a smaller range of motion. The speed of the motions is limited by the capabilities of the robot arm. A slow stair climb motion was also simulated and called Stair Climb for the purposes of this study.

In the experiments, an IMU was attached to the “thigh” and “shank” segments of the robot. The goal was to determine the orientation of the segments and the angle between them, or the “knee” angle. These IMUs were attached to the robot “thigh” and “shank” segments using custom holders designed to put the IMUs in the same place for each trial.

A simplified, hardwired data collection system was used for these experiments as telemetry was not required. For this study the IMU data was collected using a MSP430 (Texas Instruments, Inc., Dallas, Tex.) microcontroller and stored on a computer for post processing using MATLAB (The Mathworks, Inc. Natick, Mass.). Three trials were performed for all three activities. The root mean-squared-error (RMSE) between the MGAS orientation angles and the robot orientation angles were calculated.

A mathematical model of the robot was created using the Matlab (The Mathworks, Natick, Mass.) programming environment. The position and orientation of the IMUs relative to the robot segments, the robot joint angle data, and the robot segment lengths were the inputs for the model. The outputs were the position and orientation of the IMUs as well as calculated accelerometer and gyroscope “signals” used as the ground truth when determining the accuracy of the IMU signals. The calculated IMU data was used to synchronize the robot and IMU data, evaluate IMU performance and develop the algorithm used to calculate joint angles from IMU data.

Referring back to FIG. 16, the actuating of the robot was followed by collecting of encoder data from the robot at step 1604 for actuating the robot and collection of IMU data at step 1606. Thereafter, the model of the robot was used to compute “ideal” IMU data from the encoder data at step 1608. At step 1610, the “ideal” and collected IMU data was then be synchronized and/or resampled for subsequent comparisons. These comparisons include a comparison of IMU data at step 1610 and, after performing the extended Kalman filtering at step 1612 to obtain orientation information for the robot, a comparison of the orientation results at step 1614.

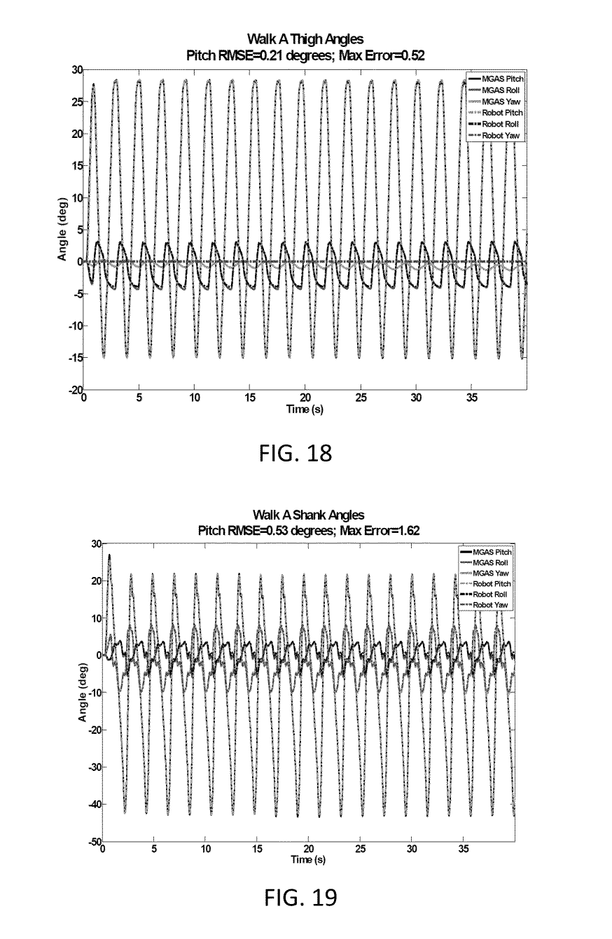

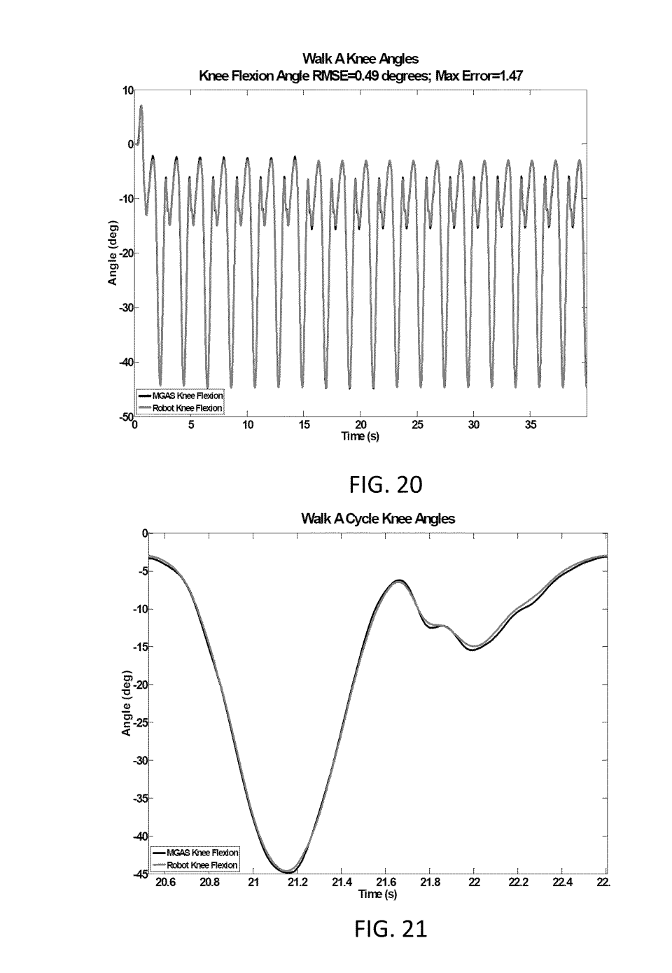

Although specific IMUs were utilized in these experiments, a range of IMU sensors were evaluated using these robotic procedures. The IMU chip used herein, that consists of a three-axis accelerometer and a three-axis gyroscope, was selected based on performance, cost, size, form factor, communication interface and ease of implementation. This chip was incorporated to an expansion board for a commercially available computer-on-module device that uses an ARM reduced instruction set processor that runs the Linux operating system, runs compiled. This configuration is substantially similar to that described above with respect to FIGS. 2, 3, and 9-10. FIGS. 18-21 show the results for Walk A. FIG. 18 shows the results of a sample trial of Walk A orientation angles from IMU data (MGAS values) and orientation angles calculated by the kinematic model (Robot values) for the “thigh” segment of the robot. FIG. 19 shows the results of a sample trial of Walk A orientation angles from IMU data (MGAS values) and orientation angles calculated by the kinematic model (Robot values) for the “shank” segment of the robot. The results of multiple strides are plotted across the horizontal axis as a function of time. The pitch RMSE and maximum pitch angle error are also displayed for this trial. FIG. 20 shows the results of a sample trial of Walking A calculated knee flexion angles (MGAS values) and knee flexion angles calculated by the kinematic model (Robot values). The results of multiple strides are plotted across the horizontal axis as a function of time. The knee flexion RMSE and maximum knee flexion angle error are also displayed for this trial. FIG. 21 shows a detailed view of a sample trial of Walking A knee flexion angles from IMU data (MGAS values) and knee flexion angles from the kinematic model (Robot values) from one simulated gait cycle. As shown in each of these figures, there is good agreement between the results based on robot encoder data (“Robot”) and the actual IMU results (“MGAS”) for all measurements and calculations.

Average results of six trials of Walk A were computed. The thigh segment pitch RMSE was 0.2 degrees (standard deviation=0.1 degrees), shank segment pitch RMSE was 0.5 degrees (standard deviation=0.0 degrees), and the knee flexion calculation RMSE was 0.5 degrees (standard deviation=0.1 degrees) with a max error of 1.5 degrees (standard deviation=0.2 degrees) of knee flexion. The RMSE of the out of sagittal plane angles were not calculated but by inspection are within one or two degrees throughout the trials.

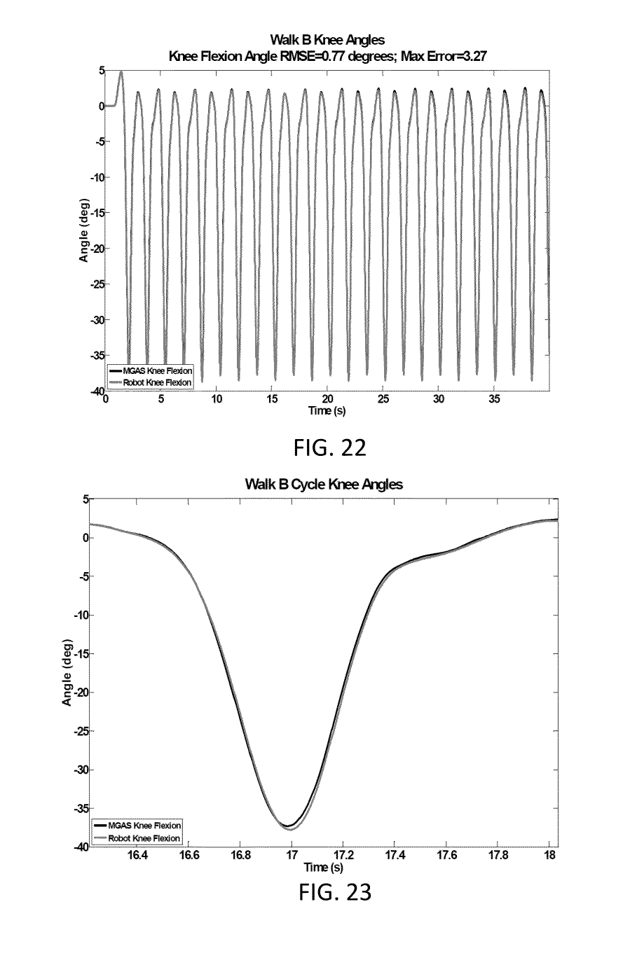

FIGS. 22 and 23 show the results for Walk B. FIG. 22 shows a sample trial of Walk B calculated knee flexion angles (MGAS values) and knee flexion angles determined by robot encoder data (Robot values). The results of multiple strides are plotted across the horizontal axis as a function of time. The knee flexion RMSE and maximum knee flexion angle error are also displayed for this trial. FIG. 23 shows an enlarged view of sample trial of Walk B knee flexion angles from IMU data (MGAS values) and knee flexion angles from the kinematic model (Robot values) from one simulated gait cycle. There is good agreement between the results based on robot encoder data (“Robot”) and the actual IMU results (“MGAS”) for all measurements and calculations.

The average results of the three trials of Walk B were the thigh segment pitch RMSE was 0.1 degrees (standard deviation=0.0 degrees), the shank segment pitch RMSE was 0.3 degrees (standard deviation=0.1 degrees) and the knee flexion RMSE was 0.8 degrees (standard deviation=0.2 degrees). Similar to the Walk A data, the out of sagittal plane orientation data appeared to be within one or two degrees by visual inspection of the plots.

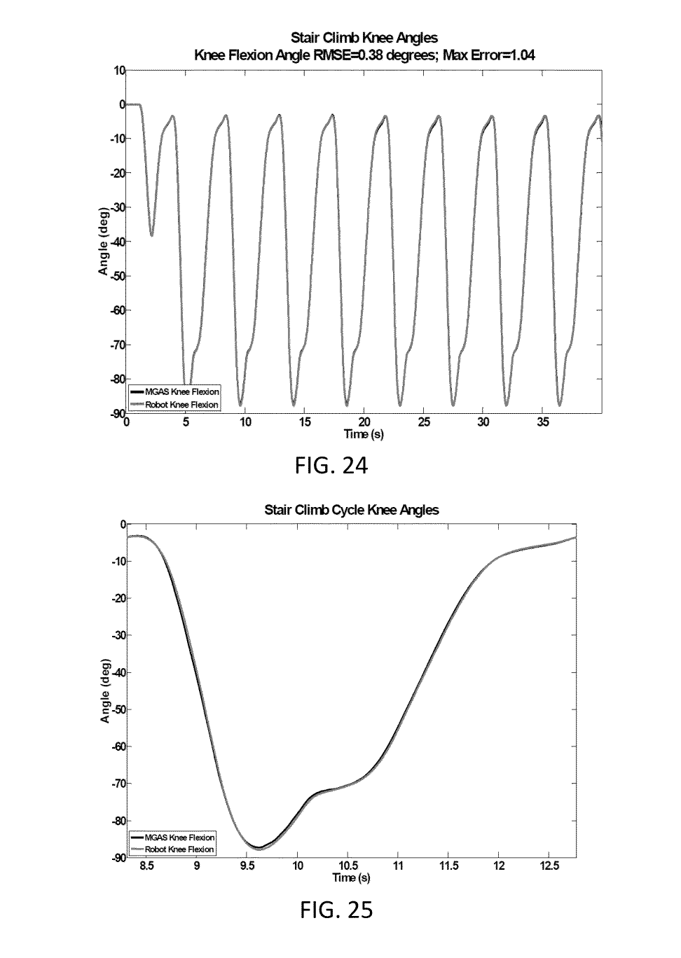

FIGS. 24 and 25 show the results for the Stair Climb activity. FIG. 24 shows a sample trial of Stair Climb calculated knee flexion angles (MGAS values) and knee flexion angles calculated by the kinematic model (Robot values). The results of multiple strides are plotted across the horizontal axis as a function of time. FIG. 25 shows an enlarged view of a sample trial of Stair Climb knee flexion angles from IMU data (MGAS values) and knee flexion angles from the kinematic model (Robot values) from one simulated gait cycle. Again, there is good agreement between the results based on encoder data (“Robot”) and the actual IMU results (“MGAS”) for all measurements and calculations.

The average slow Stair Climb activity segment pitch RMSE for the thigh segment was 0.3 degrees (standard deviation=0.1 degrees), for the shank segment was 0.1 degrees (standard deviation=0.1 degrees) and for knee flexion 0.4 degrees (standard deviation=0.0 degrees). Similar to the previous two activities the error of out of sagittal plane motion appeared to be within a few degrees by visual inspection of the data.

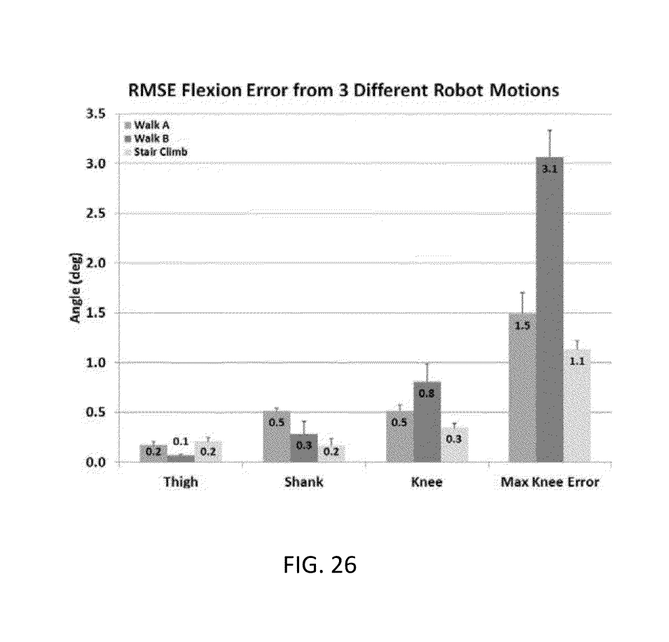

The average maximum knee flexion errors per trial were 1.5 degrees (standard deviation=0.2 degrees), 3.1 degrees (standard deviation=0.2 degrees) and 1.2 degrees (standard deviation=0.3 degrees) for Walk A, Walk B and Stair Climb activities, respectively. The flexion error from the different robot motions are shown in FIG. 26. The maximum knee error bars represent the maximum discrepancy between the encoder data based results and the MGAS system results for any two data points and is very good as compared to current methods.

As noted above, the results from these kinematic tests show good agreement and are within the accuracy requirements for gait analysis. The RMSE values for sagittal plane orientations are within 1 degree RMSE, which was the goal set for the kinematic portion of the system. The out of sagittal plane motions are also accurate to within a few degrees, however sagittal plane motions are of secondary importance for gait analysis purposes There is no additional reference for the yaw component of the limb orientation; therefore this calculation is dependent purely on the gyroscope signal and vulnerable to drift errors. This may be a factor in real world testing of the system when soft tissue artifact and inconsistent motion comes into play. However, with additional processing, a stable estimate of yaw orientation, or heading, can be made without the use of additional sensors such as magnetometers. By avoiding using magnetometers, the concern over ferrous magnetic perturbations is avoided and this system will work in any setting.

These trials demonstrate that portable, inexpensive inertial sensors can be used to accurately track complicated repeated biomechanical motion. Incorporating this into the proposed MGAS will result in a tool that will give prosthetists, clinicians and researchers more information to improve the performance of lower leg prosthesis and the overall quality of life of amputee.

For testing in a gait laboratory environment, an expansion board for these IMUs was designed for two applications. The first application is to control and manage data from a portable force/moment (F/M) foot sensor which is strapped to the bottom of the shoe, as discussed above with respect to FIGS. 9 and 10. The foot sensor consists of a toe and heel nodes incorporating load cells that isolate loads in the cardinal directions to eliminate cross talk. These nodes are capable of very accurate force measurements in three dimensions that can be resolved to propulsive or braking forces, vertical load forces (SI), anterior/posterior (AP) and medial-lateral (ML) ground reaction forces (GRF) and moments or torques in each node about a vertical axis. The toe and heel node each contain a circuit board consisting of signal conditioning and 16-bit (effective resolution of greater than 14.5 bits) analog to digital conversion (ADC) circuitry for 10 channels and the selected IMU sensor.

The second application of the expansion board is as a limb segment (e.g. thigh, shank) IMU sensor, as discussed above with respect to FIGS. 2 and 3. In this case the MSP430 microcontroller controls the IMU and on hardware clock. The battery only powers the expansion board and computer-on-module device which has the same function as the foot sensor application but only stores and transmits inertial data from the IMU on the expansion board. In both applications, the expansion board manages the battery charge/discharge with power management circuits. A means to charge the battery, and command line access to the computer-on-module device, is provided through micro-USB on the expansion board.

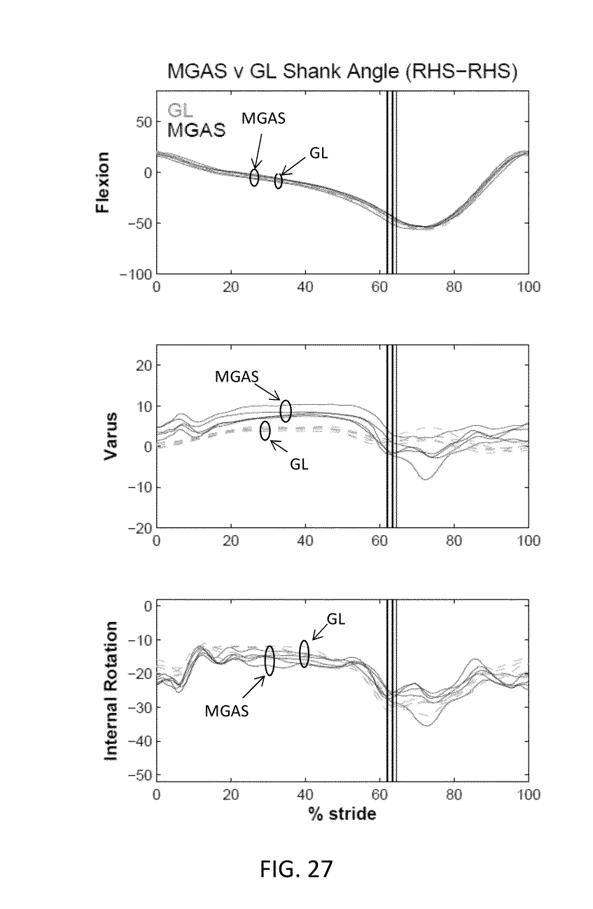

To further evaluate the effectiveness of the analysis system of the various embodiments, a gait lab study was conducted in which measurements were recorded from the system in accordance with the various embodiments (labeled “MGAS” in the following figures). Video gait lab data was recorded simultaneously and is presented for comparison purposes (labeled “GL” in the following figures). The measurements were obtained for a single, healthy subject walking at normal speed. Preliminary data from this study is presented in FIGS. 27-32.

Video gait data is show for comparison in these plots, however video gait data is an indirect measurement and should not be considered ground truth. The markers used for video gait analysis were not mounted in the same location as the inertial measurement sensors, and some variation in results is to be expected. Both methods will be subject to skin, clothing, and muscle motion artifacts, however these artifacts will be different for the two measurement techniques. Both sets of data are provided to illustrate that the results from the MGAS system are physiologically meaningful.

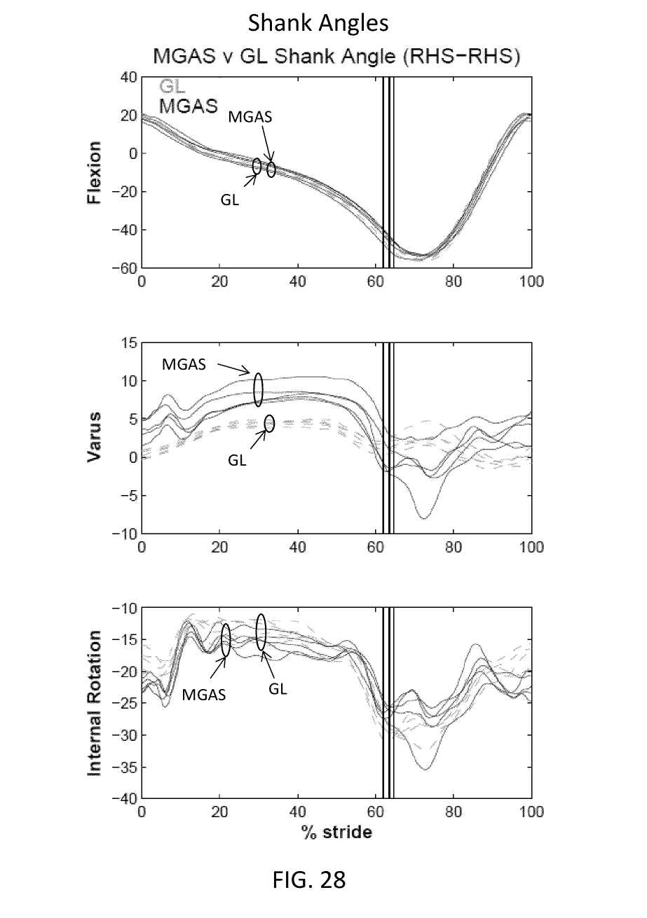

Thigh and shank angle measurements are compared in FIGS. 27 and 28. FIG. 27 shows plots of angle in three physiological planes for the thigh. In these plots, the results for multiple gait cycles are overlayed on top of one another. The horizontal axis represents percent gait cycle with 0 and 100 representing successive heel strikes, and the vertical lines representing toe off. Portions to the left of the vertical bars represent the stance phase of the gait cycle and portions to the right of the vertical bars represent swing phase. FIG. 28 shows plots of angle in three physiological planes for the shank. For each of FIGS. 27 and 28, sagittal angles (flexion and extension rotation or pitch) presented in the first row, coronal angles (varus and valgus rotation or roll) are presented in the second row, and transverse angles (internal and external rotation or yaw) are presented in the third row. The horizontal axis is percent gait cycle or stride from heel strike to heel strike. The solid vertical lines are toe off for each of the strides. The y axis is angle.

As shown in FIGS. 27 and 28, there is generally good agreement in sagittal angle measurements (first row) for the two methods. The agreement between coronal angle measurements (second row), and transverse angle measurements (third row) for the two methods is sufficient for physiological significance determinations to be performed. Motion capture video gait analysis systems are most accurate in the sagittal plane. Video system measurements out of the sagittal plane have been reported to be inaccurate in these systems due to intra and interoperator inconsistencies and the small magnitudes of the angle being measured. The differences reported between the MGAS and standard gait laboratory system are par with the accuracy reported for the standard system. Moreover, the measures that clinicians are most interested in are in the sagittal plane.

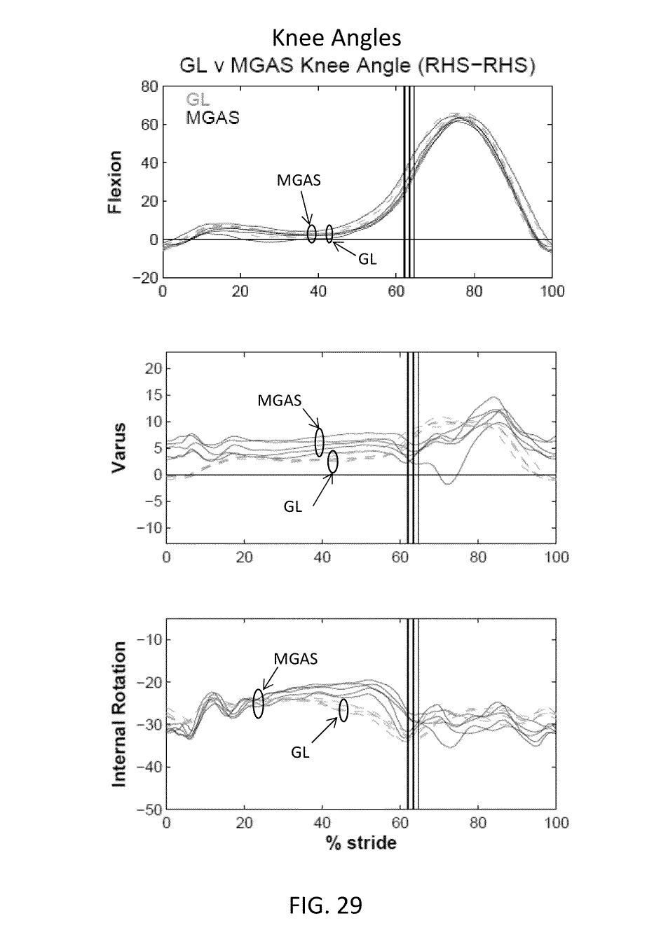

Knee angle measurements are compared in FIG. 29, where the knee angle is obtained from a combination of the thigh and shank data. The results for multiple gait cycles are overlayed on top of one another. The horizontal axis represents percent gait cycle with 0 and 100 representing successive heel strikes, and the vertical lines representing toe off. Portions to the left of the vertical bars represent the stance phase of the gait cycle and portions to the right of the vertical bars represent swing phase. FIG. 29 shows plots of angle in three physiological planes for the knee (sagittal, coronal, and transverse), as described above with respect to FIGS. 27 and 28. FIG. 29 shows the measurements for the individual strides, similar to FIGS. 27 and 28.

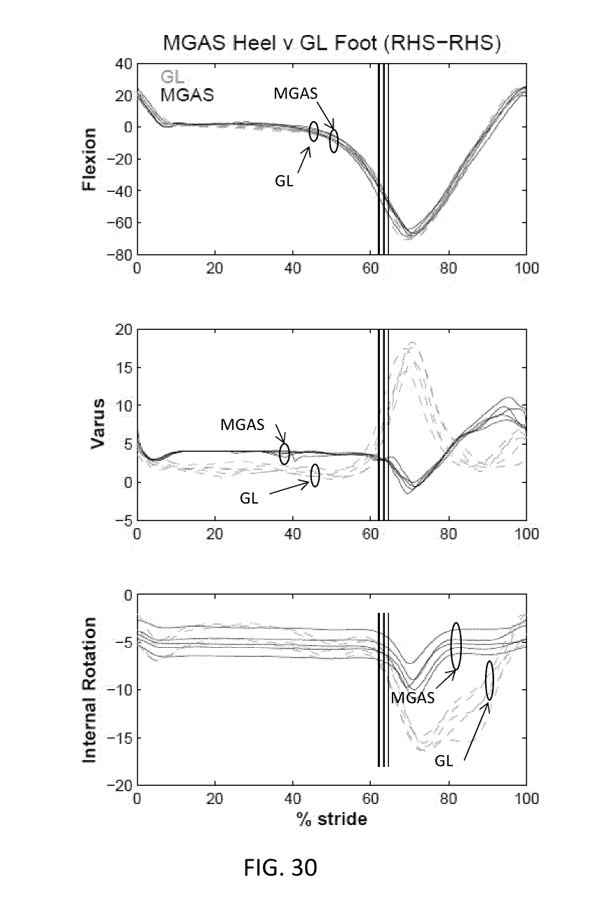

As shown in FIGS. 29 and 30, there is generally good agreement in sagittal angle measurements (first row) and sufficient agreement between coronal angle measurements (second row) and transverse angle measurements (third row). The differences in these measurements are attributed to differences in location of the markers for the two methods, artifacts due to skin, muscle, and clothing motion.

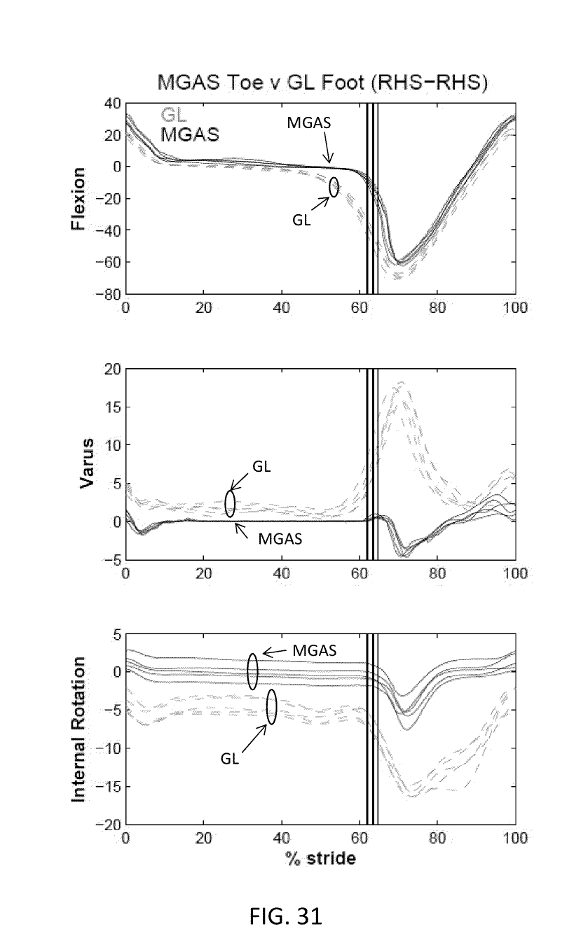

Heel and toe orientation measurements of the foot are compared in FIGS. 30 and 31. For the MGAS system, the orientation measurements are made in heal and toe units of the foot pads underneath the shoe, whereas the video gait analysis reflective markers were mounted on top of the shoe. Video motion analysis data for the foot will be more subject to artifacts in these results, and discrepancies between the two techniques are to be expected. FIGS. 30 and 31 shows plots of angle in three physiological planes for the heel and toe, respectively (sagittal, coronal, and transverse), as described above with respect to FIGS. 27 and 28. As shown in FIGS. 30 and 31, there is good agreement in sagittal angle measurements (first row), but expected differences occur between coronal angle measurements (second row) and transverse angle measurements (third row) as the MGAS system is capable of measuring the flexion of the foot, whereas the gait lab data cannot account for the effects of foot flexion.

The standard gait laboratory measures the orientation of the foot as one body whereas the MGAS system measures the orientation of the heel and forefoot. The data are presented together for illustration only, and comparison should not be made due to these differences in measurement technique. As expected, the gait laboratory foot results are more similar to the MGAS heel measurements as compared to the forefoot.

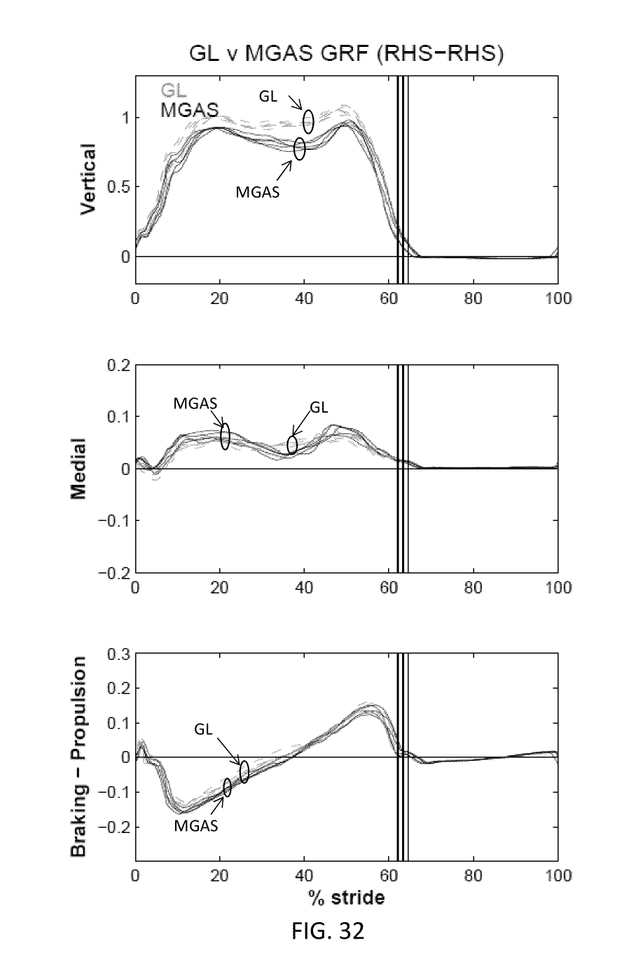

FIG. 32 shows measurements of ground reaction forces. In particular, these include vertical load, medial or lateral loads, and braking (propulsion) ground reaction forces. The results for multiple gait cycles are overlayed on top of one another. The horizontal axis represents percent gait cycle with 0 and 100 representing successive heel strikes, and the vertical lines representing toe off. Portions to the left of the vertical bars represent the stance phase of the gait cycle and portions to the right of the vertical bars represent swing phase. For the system of the various embodiments, the ground reaction force measurements were obtained by combining the force data from the heel and toe foot sensors with the orientations from FIGS. 30 and 31 to transform the forces back into the ground reference frame to match the force plate data of the gait laboratory. FIG. 32 is the data for all the individual strides. The first row is the vertical force, the second row is the medial and lateral force and the third row is the force in the anterior and posterior direction or in the direction of travel. The data for the video gait laboratory system is incomplete since it excludes data for foot strikes that did not completely hit the force plate that is embedded in the floor. Thus, this highlights an advantage of the system of the various embodiments in that every foot strike is measured. As shown in FIG. 32, the data collected is in good agreement,

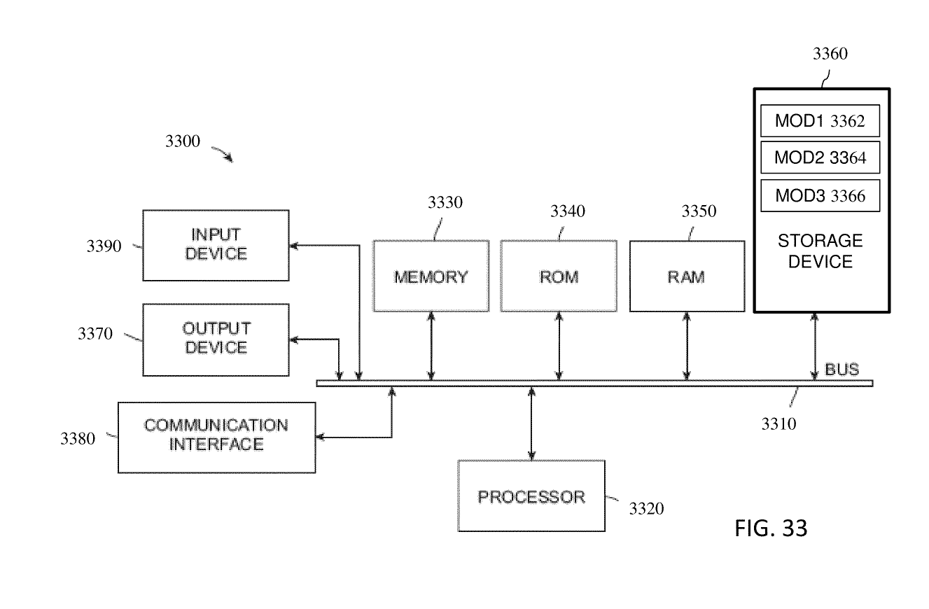

Various embodiments of the present technology are carried out using one or more computing devices. With reference to FIG. 33, an exemplary system 3300 includes a general-purpose computing device 3300, including a processing unit (CPU or processor) 3320 and a system bus 3310 that couples various system components including the system memory 3330 such as read only memory (ROM) 3340 and random access memory (RAM) 3350 to the processor 3320. The system 3300 can include a cache 3322 of high speed memory connected directly with, in close proximity to, or integrated as part of the processor 3320. The system 3300 copies data from the memory 3330 and/or the storage device 3360 to the cache 3322 for quick access by the processor 3320. In this way, the cache provides a performance boost that avoids processor 3320 delays while waiting for data. These and other modules can control or be configured to control the processor 3320 to perform various actions. Other system memory 3330 may be available for use as well. The memory 3330 can include multiple different types of memory with different performance characteristics. It can be appreciated that the disclosure may operate on a computing device 3300 with more than one processor 3320 or on a group or cluster of computing devices networked together to provide greater processing capability. The processor 3320 can include any general purpose processor and a hardware module or software module, such as module 1 3362, module 2 3364, and module 3 3366 stored in storage device 3360, configured to control the processor 3320 as well as a special-purpose processor where software instructions are incorporated into the actual processor design. The processor 3320 may essentially be a completely self-contained computing system, containing multiple cores or processors, a bus, memory controller, cache, etc. A multi-core processor may be symmetric or asymmetric.

The system bus 3310 may be any of several types of bus structures including a memory bus or memory controller, a peripheral bus, and a local bus using any of a variety of bus architectures. A basic input/output (BIOS) stored in ROM 3340 or the like, may provide the basic routine that helps to transfer information between elements within the computing device 3300, such as during start-up. The computing device 3300 further includes storage devices 3360 such as a solid state hard disk drive, a magnetic disk drive, an optical disk drive, tape drive or the like. The storage device 3360 can include software modules 3362, 3364, 3366 for controlling the processor 3320. Other hardware or software modules are contemplated. The storage device 3360 is connected to the system bus 3310 by a drive interface. The drives and the associated computer readable storage media provide nonvolatile storage of computer readable instructions, data structures, program modules and other data for the computing device 3300. In one aspect, a hardware module that performs a particular function includes the software component stored in a non-transitory computer-readable medium in connection with the necessary hardware components, such as the processor 3320, bus 3310, display 3370, and so forth, to carry out the function. The basic components are known to those of skill in the art and appropriate variations are contemplated depending on the type of device, such as whether the device 3300 is a small, handheld computing device, a desktop computer, or a computer server.

Although the exemplary embodiment described herein employs the hard disk 3360, it should be appreciated by those skilled in the art that other types of computer readable media which can store data that are accessible by a computer, such as magnetic cassettes, solid state memory devices, digital versatile disks, cartridges, random access memories (RAMs) 3350, read only memory (ROM) 3340, a cable or wireless signal containing a bit stream and the like, may also be used in the exemplary operating environment. Non-transitory computer-readable storage media expressly exclude media such as energy, carrier signals, electromagnetic waves, and signals per se.

To enable user interaction with the computing device 3300, an input device 3390 represents any number of input mechanisms, such as a microphone for speech, a touch-sensitive screen for gesture or graphical input, keyboard, mouse, motion input, speech and so forth. An output device 3370 can also be one or more of a number of output mechanisms known to those of skill in the art. In some instances, multimodal systems enable a user to provide multiple types of input to communicate with the computing device 3300. The communications interface 3380 generally governs and manages the user input and system output. There is no restriction on operating on any particular hardware arrangement and therefore the basic features here may easily be substituted for improved hardware or firmware arrangements as they are developed.

For clarity of explanation, the illustrative system embodiment is presented as including individual functional blocks including functional blocks labeled as a “processor” or processor 3320. The functions these blocks represent may be provided through the use of either shared or dedicated hardware, including, but not limited to, hardware capable of executing software and hardware, such as a processor 3320, that is purpose-built to operate as an equivalent to software executing on a general purpose processor. For example, the functions of one or more processors presented in FIG. 33 may be provided by a single shared processor or multiple processors. (Use of the term “processor” should not be construed to refer exclusively to hardware capable of executing software.) Illustrative embodiments may include microprocessor and/or digital signal processor (DSP) hardware, read-only memory (ROM) 3340 for storing software performing the operations discussed below, and random access memory (RAM) 3350 for storing results. Very large scale integration (VLSI) hardware embodiments, as well as custom VLSI circuitry in combination with a general purpose DSP circuit, may also be provided.

The logical operations of the various embodiments are implemented as: (1) a sequence of computer implemented steps, operations, or procedures running on a programmable circuit within a general use computer, (2) a sequence of computer implemented steps, operations, or procedures running on a specific-use programmable circuit; and/or (3) interconnected machine modules or program engines within the programmable circuits. The system 3300 shown in FIG. 33 can practice all or part of the recited methods, can be a part of the recited systems, and/or can operate according to instructions in the recited non-transitory computer-readable storage media. Such logical operations can be implemented as modules configured to control the processor 3320 to perform particular functions according to the programming of the module. For example, FIG. 33 illustrates three modules Mod1 3362, Mod2 3364 and Mod3 3366, which are modules configured to control the processor 3320. These modules may be stored on the storage device 3360 and loaded into RAM 3350 or memory 3330 at runtime or may be stored as would be known in the art in other computer-readable memory locations.

Although the present invention has been described in considerable detail with reference to certain preferred versions thereof, other versions are possible. Therefore, the spirit and scope of the appended claims should not be limited to the description of the preferred versions contained herein.

The reader's attention is directed to all papers and documents which are filed concurrently with this specification and which are open to public inspection with this specification, and the contents of all such papers and documents are incorporated herein by reference.

All the features disclosed in this specification (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means for” performing a specified function, or “step for” performing a specific function, is not to be interpreted as a “means” or “step” clause as specified in 35 U.S.C §112, sixth paragraph. In particular, the use of “step of” in the claims herein is not intended to invoke the provisions of 35 U.S.C §112, sixth paragraph.

What is claimed is:

1. A method of performing gait analysis of a subject, comprising:
obtaining a plurality of measurement sets for a subject, each of the plurality of measurement sets comprising inertial measurements obtained from a sensor device associated with a different one of a plurality of segments of the subject;
calculating a sensor orientation for the sensor device associated with each of the plurality of segments based at least on a portion of a corresponding one the plurality of measurement sets;
computing a segment orientation for each of the plurality of segments based on a data fusion process applied at each of the plurality segments, the data fusion process comprising combining at least a one of the plurality measurement sets and the corresponding sensor orientation to estimate the corresponding segment orientation; and
determining joint angles based on the estimate of the segment orientation for each of the plurality of segments.

2. The method of claim 1, wherein the determining further comprises calculating each one of the joint angles by subtracting the pitch for each of the different segments associated the one of the joint angles.

3. The method of claim 1, wherein the computing comprises performing a Kalman filtering process.

4. The method of claim 3, wherein each of the plurality of measurement sets comprises accelerometer measurements and gyroscope measurements.

5. The method of claim 5, wherein the Kalman filtering process comprises applying a weighting to covariances associated with each of the accelerometer measurements and the gyroscope measurements during the computing based on a motion of the subject, wherein covariances associated with the gyroscope measurements are weighted more heavily if the subject is in motion, else convariances associated with the accelerometer measurements are weighted more heavily.

6. The method of claim 1, further comprising:
obtaining a plurality of additional measurement sets for the subject, the plurality of additional measurement sets comprising load and moment measurements from at least one footpad attached to a sole of a foot of the subject; and
combining the plurality of additional measurement sets to determine limb forces and joint torques for at least one of the plurality of segments.

7. The method of claim 6, wherein the plurality of the additional measurements further comprises inertial measurements for each of a forefoot and a heel of the subject, and wherein the method further comprises repeating the calculating and computing for each of the forefoot and the heel based at least on the plurality of additional measurements.

8. A system for performing gait analysis, comprising:
a processor;
a communications interface configured for receiving a plurality of measurement sets, each of the plurality of measurement sets comprising inertial measurements obtained from a sensor device associated with different one of a plurality of segments of a subject; and
computer-readable medium having stored thereon a plurality of instructions for causing the processor to perform the steps of:
calculating a sensor orientation for the sensor device associated with each of the plurality of segments based at least on a portion of a corresponding one the plurality of measurement sets,
computing a segment orientation for each of the plurality of segments based on a data fusion process applied at each of the plurality of segments, the data fusion process comprising combining at least a one of the plurality measurement sets and the corresponding sensor orientation to estimate the corresponding segment orientation, and
determining joint angles based on the estimate of the segment orientation for each of the plurality of segments.

9. The system of claim 8, wherein the determining further comprises calculating each one of the joint angles by subtracting the pitch for each of the different segments associated the one of the joint angles.

10. The system of claim 8, wherein the computing comprises performing a Kalman filtering process.

11. The system of claim 10, wherein each of the plurality of measurement sets comprises accelerometer measurements and gyroscope measurements.

12. The system of claim 11, wherein the plurality of instructions further comprises instructions for causing the process to perform the Kalman filtering process by applying a weighting to covariances associated with each of the accelerometer measurements and the gyroscope measurements during the computing based on a motion of the subject, wherein covariances associated with the gyroscope measurements are weighted more heavily if the subject is in motion, else covariances associated with the accelerometer measurements are weighted more heavily.

13. The system of claim 8, wherein the communications interface is further configured for receiving a plurality of additional measurement sets for the subject, the plurality of additional measurement sets comprising force and moment measurements from at least one footpad attached to a sole of a foot of the subject; and
wherein the plurality of instructions further comprise instructions for causing the processor to perform the step of combining the plurality of additional measurement sets in a kinematic and kinetic model to determine limb forces and joint torques for at least one of the plurality of segments.

14. The system of claim 13, wherein the plurality of the additional measurements further comprises inertial measurements for each of a forefoot and a heel of the subject, and,
wherein the plurality of instructions further comprise instructions for causing the processor to perform the step of repeating the calculating and computing for each of the forefoot and the heel based at least on the plurality of additional measurements.

15. The system of claim 8, the plurality of instructions further comprising instructions for causing the processor to determine a force on at least one of a knee, a hip, a lower leg, an upper leg, or prosthetic limb-human interface.

16. The system of claim 8, the plurality of instructions further comprising instructions for causing the processor to determine a torque on at least one of a knee, a hip, a lower leg, an upper leg, or prosthetic limb-human interface.

17. A sensor for analyzing gait and ground reaction forces, comprising:
a forefoot portion removably attachable to a sole of a subject's forefoot and comprising at least one force sensor;
a heel portion removably attachable to a sole of the subject's heel and comprising at least one force sensor; and
a processing unit communicatively coupled to the forefoot portion and the heel portion and configured for transmitting sensor signals from the forefoot portion and the heel portion to a remote computing device to perform an analysis of the gait of the subject and to compute the ground reaction forces,
wherein at least one of the forefoot portion, the heel portion, and the processing unit comprises at least one inertial measurement sensor.

18. The sensor of claim 17, wherein the at least one force sensor of the forefoot portion and the at least one force sensor of the heel portion each comprise a multi-axis load and moment sensor.

19. The sensor of claim 17, wherein at least one of the forefoot portion and the heel portion comprises the at least one inertial measurement sensor.

20. The sensor of claim 17, wherein the processing unit is removably attached to the foot of the subject and comprises the at least one inertial measurement sensor.
